(19) United States
(12) Patent Application Publication
(10) Pub. No.: US 2007/0224124 A1
(43) Pub. Date: Sep. 27, 2007

(54) Novel SH2containing inositol 5'-phosphatase isoform that partners with the Grb2 adapter protein

(75) Inventors: William G. Kerr, Tampa, FL (US); John M. Ninos, Tampa, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: University of South Florida, Tampa, FL (US)

(21) Appl. No.: 11/726,028

(22) Filed: Mar. 19, 2007

Related U.S. Application Data
(63) Continuation of application No. 10/605,452, filed on Sep. 30, 2003, now abandoned.
(60) Provisional application No. 60/319,583, filed on Sep. 30, 2002.

(51) Int. Cl. C12N 15/87; A61K 49/00; C12N 5/00; G01N 33/53; C07H 21/04

(52) U.S. Cl. 424/9.1; 435/375; 435/377; 435/461; 435/7.21; 536/23.1

(57) ABSTRACT

SH2 containing inositol 5′-phosphate (SHIP) modulates the activation of immune cells after recruitment to the membrane by Shc and the cytoplasmic tails of receptors. A novel SHIP isoform of approximately 104 kd expressed in primitive stem cell populations (s-SHIP) is described. It was found that s-SHIP is expressed in totipotent embryonic stem cells to the exclusion of the 145-kd SHIP isoform expressed in differentiated hematopoietic cells. s-SHIP is also expressed in primitive hematopoietic stem cells, but not in lineage-committed hematopoietic cells. In embryoic stem cells, s-SHIP partners with the adapter protein Grb2 without tyrosine phosphorylation and is present constitutively at the cell membrane. It is postulated that s-SHIP modulates the activation threshold of primitive stem cell populations.

### CROSS REFERENCE TO RELATED APPLICATIONS

This application claims priority to U.S. Provisional Patent Application Ser. No. 60/319,583 filed Sep. 30, 2002, the specification

### FEDERAL RESEARCH STATEMENT

Federal Research Statement Paragraph] NIH/NIDDK RO1 DK54767

### SUMMARY OF INVENTION

The present invention is a method of cloning a novel SHIP isoform, s-SHIP, and showing that it is expressed in murine embryonic stem cells and murine hematopoietic stem cells. Because of this it may be expressed in all stem cell types—ES, mesnechymal neural, hepatic, neural, muscle, and the like.

Its function has been partially characterized and it is recruited to the cell membrane even without an acute stimulation of ES cells, it can access its substrate and modulate PIPS levels that accumulate from P activation in response to basal level growth factor stimulation, even in resting stem cells—thus s-SHIP is likely to influence the proliferation and differentiation of stem cells.

Advantages of the present invention include: (1) human s-SHIP/SIP1 10 as a marker of stem cells an assay for expression of s-SHIP could be used determine whether a putative human ES cell line is really an ES cell by antibodies or RNA detection of s-SHIP or detection of its enzymatic activity; (2) pharmaceutical modulation of s-SHIP/SIP110 to control the growth and differentiation of human ES cells or other stem cell populations; (3) the human SIP110 cDNA—enforced expression, or inducible expression of s-SHIP could be used to keep human stem cells in an undifferentiated state; (4) the mouse s-SHIP cDNA if mouse cDNA can replace the human cDNA, then enforced expression, or inducible expression of murine s-SHIP, could be used to keep human stem cells in an undifferentiated state; and (5) fluorogenic assays with enzymatic substrates that can be carried out on viable cells to allow PACS sorting of primitive stem cell populations—either embryonic or tissue stem cells.

The inventive method allows for identifying the presence, and delineation, of embryonic stem cells in a cell sample comprising a mixture of cells, the method comprising the steps of: adding to the sample M-MLV reverse transcriptase to primer pairs selected from the group consisting of s-SHIP, SHIP, and β-actin; and performing nested RT-PCR reactions wherein the presence of embryonic stem cells in the sample is indicated by cells that express s-SHIP mRNA. The method is applied to determine the presence of embryonic stem cells in the sample by the presence of s-SHIP enzymatic activity. Another indicator of the presence of the s-SHIP enzyme is achieved by adding anti-ship monoclonal antibody P2C6 to the sample under conditions, and for a time, suitable for antibody binding and detecting the presence of the antibodies bound to the cells in the sample. Detection can be achieved by labeling the antibodies with a flourochrome and testing by fluorescent activated cell sorting. The inventive method is also applicable for identifying the presence, and delineation, of hematopoietic stem cells in a cell sample comprising a mixture of cells, the method comprising substantially the same steps used for detection in embryonic stem cells. The method is also applicable to identifying subcellular signaling complexes involved in stem cell function co-localization by confocal immunofluorescent microscopy.

In addition to its diagnostic functionality, the method is also applicable therapeutically. Stem cells can be manipulated, due to s-SHIP”s competition with mSosI, to either induce or retard cellular proliferation and differentiation. For example, one method of inhibiting differentiation of step cells comprises the step of contacting the cells with s-SHIP enzyme whereby the accumulation of the s-SHIP enzyme prevents the accumulation of PI³,⁴,⁵P₃. Generally, the stem cells are selected from a group consisting of hematopoietic progenitors of mature blood cells, hematopoietic progenitors of lymph cells, and embryonic stem progenitor cells.

The invention does not only consist of the s-SHIP enzyme, but includes a chemically inducible nucleic acid promoter fragment isolated from the 5″ flanking region upstream of the coding region of the SHIP gene.

This promoter fragment is found in murine and human embryonic, as well as hematopoietic stem cells. By implanting a target cell population with cellular hosts of the nucleotide sequence, the vector cells can be used as a negative regulator of cell activation in populations such as B-Lymphoid, myeloid, and mast cells. Possible unicellular host cells could be a culture or tissue culture of cells selected from the group consisting of, a murine embryonic stem cell, a murine hematopoietic stem cell, a human embryonic stem cell, or a human hematopoietic stem cell.

The invention also consists of a method for inducing proliferation of stem cells by introducing anti-SHIP shRNA into the cell by electroporation. Additionally a means whereby identification stem cells in a cell sample is possible via detection of s-SHIP by immunofluorescence. This can be accomplished where the cell sample is a mixture of cells, in vivo, or in any situation where the presence of s-SHIP can be indicated. Also a method of identifying subcellular signaling complexes involved in stem cell function co-localization by confocal immunofluorescent microscopy is provided.

Proliferation of stem cells can also be induced by the introduction of an inhibitor of s-SHIP activity, such as a dominant negative mutant.

### BRIEF DESCRIPTION OF DRAWINGS

For a fuller understanding of the nature and objects of the invention, reference should be made to the following detailed description, taken in connection with the accompanying drawings, in which:

FIG. 1 is an illustration of how ES cells express e SHIP mRNA species different from that expressed in mature hematopoietic cells.

FIG. 2 indicates, graphically, the nucleotide sequence of s-SHIP cDNA and the predicted amino acid sequence of its major open reading frame (ORF).

FIG. 3 shows the organization of the first exon of s-SHIP.

FIG. 4 graphically indicates the analysis of s-SHIP and SHIP mRNA expression in ES cells and hematopoiesis.

FIG. 5 blot analysis revealing that HSCs express s-SHIP mRNA.

FIG. 6 illustrates that ES cells express the 8-SHIP protein isoform that associates with the Grb2 adapter protein.

FIG. 7 depicts the organization of the first axon of SIP-110, the human homolog of s-SHIP.

FIG. 8 demonstrates the decrease in s_SHIP expression after electroporation of embryonic stem cells with anti-SHIP shRNA.

### BRIEF DESCRIPTION OF SEQUENCES

Sequence 1 is a mouse s-SHIP sequence of the SHIP isoform.

Sequence 2 is a mouse s-SHIP sequence of the SHIP isoform.

Sequence 3 is a human s-SHIP sequence of the SHIP isoform.

Sequences 4-8 are mouse s-SHIP promoter regions.

Sequences 9-10 are human S-SHIP promoter regions.

### DETAILED DESCRIPTION

Cell Culture

The ES cell line, TLI, was obtained from Dr Patricia A. Labosky (University of Pennsylvania, Philadelphia) and was cultured in Dulbecco modified Eagle medium (Gibco-BRL, Grand Island, N.Y.) supplemented with 15% fetal bovine serum (FBS) (Summit Labs, Fort Collins. Colo.), 0.1 mM MEM nonessential amino acids, 2 mM L-glutamine, 50 μ/mL gentamicin, 50μ β-mercaptoethanol (β-ME), and 1000 U/mL leukemia inhibitory factor (LIF) (Gibco-BR.L). The ES cell line, EI0, was derived from TLI. WEHI-23 1, 70Z/3, A20, and BAL7 (B-cell lines) were maintained in RPMI 1640 medium (Mediatech, Herndon, Va.) with 10% FBS, 2 mM L-glutamine, 50 μM β-ME (Gibco-BRL), and antibiotics. Hepa 1-6 and 293T cell lines were maintained in DMEM medium with the same supplements as described for the RPMI medium.

Reverse Transcription Polymerase Chain Reaction

Cells were washed once with ice-cold phosphate-buffered saline (PBS) and pelleted. Then cells were lysed, and total RNA was prepared using the RNeasy Mini Kit (Qiagen, Valencia, Calif.) according to the manufacturer”s instructions. Reverse transcription (RT) of total cellular RNA and amplification of cDNA products for specific gene sequences was carried out using Ready-to-Go RT-PCR Beads (Ainersham Pharmacia, Piscataway, N.J.) following the manufacturer”s instructions. In this series of RTpolymerase chain reaction (PCR), 1-μg total RNA and 1-μ each gene-specific primer (10 mM) were placed in a 50 μL reaction volume. In general, PCR conditions were as follows: denaturation for 30 seconds at 94° C., annealing for 30 seconds at a temperature optimal for the primer pair (see below), and a I-minute extension at 72° C. Nested RT-PCR assays to detect mRNAs expressed in sorted cells were carried out as described below.

Cell Sorts and Nested RT-PCR Assays

Femurs and tibias from adult (4- to 6-month-old) male C57BL/6 mice were isolated. Adult bone marrow (ABM) was flushed into chilled RPMI 1640 medium. Fetal livers (FLs) (day 14.5 of gestation) were isolated from pregnant female C57BL/6 mice. Cells were teased from the FLs into chilled RPMI. Cell clusters from the ABM and FL preparations were dispersed into single-cell suspensions with transfer pipettes and were passed over 70μ nylon cell strainers. Cell suspensions were pelleted, and red blood cells (RBCs) were lysed for 5 minutes at 4° C. in RBC lysis buffer (163 mM NH. 10 mM KHCO3, 0.13 mM EDTA). After RBC lysis, the cells were washed in PBS, passed over 70 μm strainers, counted, and resuspended in staining media (PBS with 3% FBS and 10 mM HEPES) at 10⁷ cells/50 μL. Cells were incubated in Fc Block (Pharmingen, San Diego, 10⁷ cells/50 μL staining media and stained with the appropriate panel of fluorochrome-conjugated antimurine antibodies at 1 to 2 μg/10⁷ cells/50 μL staining media. Antibodies used were a lineage-fluorescein isothiocyanate panel of Mac-1 (Caltag Laboratories, Burlingame, Calif.), Gr-I, CD3, CD4 (GKI.5), CD8a (53-6.7) B220 (Pharmingen); B220-phycoerythrin (PE), Terl I 9-PE, c-kit-PE, and biotinylated Sca-1 (Pharmingen). Biotinylated Sca-I was revealed with streptavidin-allophycocyanin (APC) (Pharmingen). After staining, the cells were washed and resuspended in staining media (50 μL/10⁷ cells) plus propidium iodide (P1) at 1.0μ for dead cell exclusion. HSCs (Sca-1+c-kit+Lin−), B-lymphoid cells (B220+Gr-1−Mac-1−), myeloid cells (Gr-1+Mac-1+B220−), and erythroid cells (Ter119+Lin−) were sorted from ABM and FL using a FACStar flow cytometer (Becton Dickinson, San Jose, Calif.) equipped with an automatic cell deposition unit. ES cells (s-SHIPpositive control), WEHI-231 cells (SHIP-positive control), and Hepa 1-6 cells (negative control) were also prepared in staining media and P1(10⁷ cells/50 μL). Fifty viable cells of each population were deposited into separate wells of a 96-well PCR plate containing lysis buffer (0.4% Nonidet P-40, 60 μM dNTP, 25 μM dithiothreitol, 0.5 U/μL RNasin [Promega, Madison, Wis.]) and were lysed for 15 minutes on ice (adapted from Hu et al). RNA was reverse transcribed using multiple primer pairs for β-actin, s-SHIP, and SHIP and 48 U M-MLV reverse transcriptase per reaction in the buffer provided (Gibco-BRL). First-round PCR of35 cycles was performed with addition of 40μ buffered RedTaq DNA polymerase (Sigma, St Louis, Mo.). One-microliter aliquots of the first-round PCR product were further amplified for 35 cycles using fully nested primers for each gene. Aliquots of second-round products were subjected to gel electrophoresis and visualized by ethidium bromide staining. cDNA and genomic cloning To determine the 5″ and 3″ termini of the s-SHIP cDNA, the inventors performed rapid amplification of cDNA ends (RACE) cloning using the Smart Race cDNA Amplification Kit (Clontech, Palo Alto, Calif.). Touch down PCR amplification conditions were used for 5″ and 3″ RACE reactions with the following cycling conditions: 30 seconds at 94° C. and 4 minutes at 72° C. for cycles 1 to 5; 30 seconds at 94° C., 30 seconds at 70° C., 4 minutes at 72° C. for cycles 6 to 10; 30. seconds at 94° C., 30 seconds at 68° C., 4 minutes at 72° C. for cycles 11 to 35. Smart Race products were cloned using the TOPO TA cloning kit (Invitrogen, Carlsbad, Calif.). Sequences derived from the termini of the 5″ and 3″ RACE clones of s-SHIP were used to design primers that amplify the entire s-SHIP cDNA by RT-PCR. Full-length s-SHIP cDNA5 were obtained by 2 separate RT-PCR amplifications of overlapping fragments that contained either 5″ or 3″ termini. These 2 fragments were cloned into the pcDNA3 vector (Invitrogen). Genomic sequences were isolated using a mouse GenomeWalker Kit (Clontech). Conditions for touchdown PCR amplification of genomic sequences were as described above. Sequencing of s-SHIP cDNAs and associated genomic clones were carried out at the DNA Sequencing Facility of the University of Pennsylvania Comprehensive Cancer Center.

Sequence Analysis

DNA and protein sequences were analyzed with MacVector 7.0 (Oxford Molecular, Madison, Wis.). Sequence alignments and comparisons were performed with the ClustalW algorithm. “The Celera Human Genome Unassembled Fragments database (http://publicstion.celera.com) (Celera Genomics, Rockville, Md.) was accessed to perform BlastN queries of the first 120 nucleotides of human 110-kD signaling inositol polyphosphate 5″-phosphatase (SIP-110) cDNA (GenBank accession number 050040).

Northern Blot Analysis and Ribonuclease Protection Assay

PolyA+RNA was prepared as described above. Northern blot analysis of SHIP mRNA expression was as described previously. For ribonuclease protection assay (RPA), the target sequence contained the 44-nucleotide SSR of s-SHIP mRNA and nucleotides 45-247 of s-SHIP (nucleotides 785-987 of SHIP). It was amplified using PCR with primers XHOESHIPI (sense) and ASHIP987 (anti-sense). This PCR product was TA-cloned into pCR2.1-TOPO (Invitrogen) and then subcloned in a reverse orientation downstream of the T3 promoter of the pRRI-amp-18 vector (Ambion, Austin, Tex.). The vector was linearized for s-SHIP/SHIP probe synthesis. Anti-sense RNA probe was synthesized and labeled with 32 using the Maxiscript T7/T3 kit (Ambion) following the manufacturer”s instructions. The probe for mouse GAPDH had 25 times lower specific activity than the s-SHIP/SHIP probe. The 100 nucleotide RNA markers were generated using templates provided by Ambion. RPA assay was performed on 10 μg total RNA for each sample and 20,000 cpm for each probe using the RPAIII kit (Ambion) according to the manufacturer”s instructions. RPA products were visualized by running Quick Point precast gels (6% polyacrylamide denaturing gel; Novex, San Diego, Calif.) and then by autoradiography.

Transfections

SHIP cDNA was kindly provided by Dr Larry Rohrschneider. SHIP and s-SHIP cDNA5 were cloned separately into the pcDNA4/HisMaxC expression vector (Invitrogen). These pcDNA4/SHIP and pcDNA4/s-SHIP plasmids were transfected into 293T kidney cells using the FuGENE 6 transfection reagent (Roche Molecular Biochemicals, Indianapolis, Ind.) according to the manufacturer”s instructions.

Antibodies

Purified mouse antiSHIP monoclonal antibody, P2C6, was obtained from Drs David Lucas and Larry Rohrschneider. It was used for immunoprecipitation at a concentration of 4 μl/1 mg total protein and for immunoblotting at a dilution of 1:1000. Rabbit antiGrb2 polyclonal antibody (sc-255; Santa Cruz Biotechnology, Santa Cruz, Calif.) was used for immunoprecipitation at a concentration of 2 jig/I mg total protein and for immunoblotting at a dilution of 1:500. Rabbit antiShc polyclonal antibody (06-203; Upstate Biotechnology, Lake Placid, N.Y.) was used for immunoblotting at a concentration of I μg/mL. Rabbit antimSosI polyclonal antibody (06-246; Upstate Biotechnology) was used for immunoblotting at a concentration of I μg/mL. Mouse antiphosphotyrosine monoclonal antibody (4G1 0; 05-321; Upstate Biotechnology) was used for immunoblotting at a concentration of 1 μg/mL.

Immunoprecipitations and Immunoblots

Approximately 10⁷ cells were washed once with ice-cold PBS and pelleted. Cell pellets were then lysed in 0.5 to 1.0 mL modified RIPA buffer (1% Nonidet P-40, 50 mM Tris-HCl [pH 7.4], 0.25% Na-deoxycholate, 150 mM NaCl, 1 mM EDTA, 1 mM Na3V04, 1 mM phenylmethylsulfonyl fluoride, 10 μg/mL leupeptin, 10 μg/mL pepstatin, 10 μg/mL aprotinin). Lysates were rocked for 30 minutes at 4° C., and cellular debris was removed by centrifugation at 14,000 g for 15 minutes. Total protein concentration of each lysate was determined by spectrophotometry using the BCA Protein Assay Kit (Pierce Chemical, Rockford, Ill.). Lysates were stored at 80° C. Whole cell lysates were subsequently mixed with NUPAGE 4× lithium dodecyl sulfate (LDS) sample buffer and lox sample reducing agent, heated for 10 minutes at 70° C., and loaded Onto NuPAGE 10% or 4% to 12% Bis-Tria 1.0 mm gels (Invitrogen). For immunoprecipitations, equal concentrations of total protein (either 500 μg in 500 μL or 1 mg in 1 mL lysis buffer) were used for each individual experiment. Samples were precleared with 0.25 μg of the appropriate control IgG (normal mouse or rabbit IgG; Santa Cruz Biotechnology) together with 50 μL appropriate agarose bead conjugate (Protein G-Agarose, Fast Flow [immunoprecipitation (I.P.)]; Protein A-Agarose, Fast Flow [1.P.]; Upstate Biotechnology) for 1 hour at 4° C. Precleared samples were transferred to fresh tubes. Four microliters P2C6 antiSHIP monoclonal antibody or 2 μg antiGrb2 polyclonal antibody were added per milligram total protein and were incubated overnight with rocking at 4° C. Fifty microliters appropriate agarose bead conjugate was then added and rocked for 2 hours at 4° C. Agarose bead conjugates were pelleted (14000 g, 5 seconds), washed 3 times with cold PBS, and boiled in NuPAGE 4× LDS sample buffer with IOX sample reducing agent for 5 minutes at 100° C. Beads were pelleted and equal volumes of the immunoprecipitate supernatant were loaded onto NuPAGE 10% or 4% to 12% Bis-Tris gels. Proteins were separated for 50 minutes at 200 V using MOPS sodium dodecyl sulfate running buffer in an XCell II Mini-Cell unit (Protein molecular weights were compared using Novex MultiMark protein standards. Separated proteins were transferred to polyvinylidene difluoride (Bio-Rad Laboratories, Hercules, Calif.) or nitrocellulose [blots] (Millipore, Bedford, Mass.) membranes for 75 minutes at 30 V using the XCell II Blot Module. Immunoblotting was carried out using the enhanced chemiluminescence Western blotting analysis system following the manufacturer”s instructions (Amersham Pharntacia).

Cell Stimulation

ES-TLI cells were split into 4 different 100-mm TC plates and grown overnight at 37° C. in ES media supplemented with 1000 U/mL LIF to approximate 80% confluence. LIF-supplemented media was removed, cells were washed twice with PBS, media without LIF was added, and cells were incubated for 5 hours. Cells were washed once with PBS and twice with Hanks balanced salt solution (HBSS; Mediatech). Then they were pre incubated in HBSS at 37° C. for 10 minutes. Cells were stimulated by the addition of HBSS containing 2000 U/ml. LIF and by incubation at 37° C. for either 2, 5, or 10 minutes. A control ES plate (0 minute) containing HBSS without LIF was incubated at 37° C. in parallel for 10 minutes. Stimulations were stopped by the removal of buffer and the addition of 10 μL ice-cold PBS/I mM Na PBSINa was removed, and 10 mL ice-cold modified RIPA buffer was added immediately. Cells were scraped, and cell lysates were processed as described above. A20 cells were grown in RPMI and were washed once with PBS and twice with HBSS. Then 2×10⁷ cells were placed in separate 15-mL conicals and were pre-incubated in HBSS at 37° C. for 10 minutes. Cells were spun down, and HBSS was removed. After that, cells were stimulated with I mL HBSS containing 20 μg/mL goat antimouse IgG (Southern Biotechnology Associates, Birmingham, Ala.) and were incubated at 37° C. for 5 minutes. Control A20 cells (0 minute) containing HESS without antimouse IgG were incubated in parallel. Stimulations were stopped by placing the conicals on ice and adding 2 mL ice-cold PBS/Na3V04 Cells were pelleted, supernatant was removed, and 1 mL ice-cold modified RIPA buffer was added. Total protein (1.0 mg in I ml.) of each preparation was used for subsequent immunoprecipitation experiments.

Membrane and Cytosol Fractionation

Two confluent 150 mm×25 mm plates of ES-TLI cells cultured in LIF were washed twice with chilled PBS, then collected with a chilled cell scraper. Cells were pelleted by centrifugation at 300 g and resuspended in 2 ml. chilled sonication buffer (20 mM Tris-HCl, pH 8, 137 mM NaCl, 10% glycerol, 2 mM EDTA, 1 mM NaVO4, 1 mM phenylmethylsulfonyl fluoride, 10 μg/mL leupeptin, 10 μg/mL pepstatin, and 10 μg/mL aprotinin). This suspension was sonicated on ice for 20 seconds at 20% full output using a Fisher Model 60 Sonic Dismembrator equipped with a one-eighth inch diameter tip (Fisher Scientific, Pittsburgh, Pa.). Trypan blue dye exclusion and examination by phase-contrast microscopy con firmed that no intact cells remained. The suspension was centrifuged at 500 g for 5 minutes at 4° C. to remove the nuclear fraction. Postnuclear supernatant was centrifuged in Beckman 15×31 mm polycarbonate tubes at 100 000 g for 20 minutes at 4° C. in a Beckman TL-I00 centrifuge equipped with a TLA-100.3 rotor. The supernatant cytosol fraction was removed, and Triton X-100 was added to a final concentration of 1%. The membrane pellet was rinsed once with chilled sonication buffer and then resuspended in chilled sonication buffer plus 1% Triton X-100. Resuspension was achieved by needle probe sonication on ice for 30 seconds at 20% full output, and the resuspended membrane fraction was allowed to solubilize at 4° C. for 45 minutes with frequent gentle vortexing. Insoluble material was removed by centrifugation at 10 000 g for 15 minutes at 4° C. ES-TLI cells from a third 150 mm plate were collected in parallel with the cells collected for fractionation in order to prepare an unfractionated whole cell lysate for comparison purposes. These cells were lysed in 1 mL of chilled sonication buffer/1% Triton X-100, rocked for 45 minutes at 4° C., and centrifuged at 10 000 g for 15 minutes at 4° C. to remove cellular debris. Total protein concentrations of the whole cell lysate, cytosol, and membrane fractions were determined by spectrophotometry using the BCA Protein Assay Kit. Whole cell lysate, cytosol, and membrane fractions were then diluted in sonication buffer/1% Triton X-100 to a final concentration of 1 mg/mL. Fractions were stored at −80° C. and 1 mg total protein (in 1 mL) of each preparation was used for subsequent immunoprecipitation experiments.

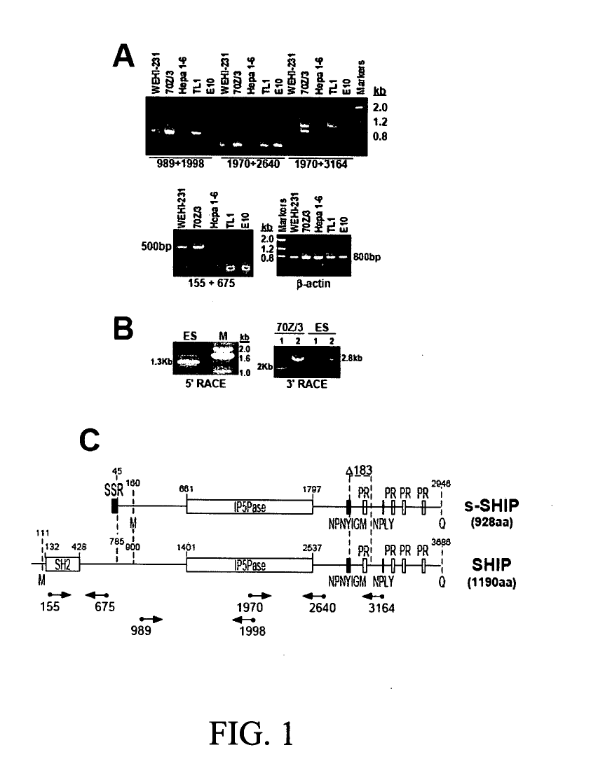

FIG. 1. illustrates how ES cells express e SHIP mRNA species different from that expressed in mature hematopoletic cells. (A) RT-PCR analysis of SHIP expression in ES cells and lineage-committed hematopoiletic cell lines. All primers used in the RT-PCR assays were designed to amplify regions of SHIP mRNA encoded by separate axons; hence, the expected amplification products must arise from amplification of cDNA, not from contaminating genomic DNA. The following cell types were analyzed: B-lymphocyte cell lines 70Z/3 and WEHI-231, hepatoma cell line Hepa 1-6 (negative control), and ES cell lines TL1 and E10. Amplification of β-actin was included 888 control for the RNA isolation and cDNA synthesis steps. PCR products were resolved on a 1% agarose gel and stained with ethidium bromide. (B) RACE cloning of s-SHIP mRNA. PCR products representing the 5″ or 3″ termini of the s-SHIP mRNA expressed in ES cells were generated by 5″ and 3″ RACE, respectively. For 3″ RACE cloning of SHIP mRNA species in 70Z13 and ES cells, the primers SHIP2766 (sense; lane 1) or SHIP1970 (sense; lane 2) were used. (C) Schematic depiction of the structure of the s-SHIP and SHIP cDNAs. Nucleotide numbering refers to GenBank accession numbers AF184912 and U52044, respectively. Functional do mains and sequence motifs encoded In the s-SHIP and SHIP mRNAs are s-SHIP Region (SSR), Src-homology 2 domain (SH2), proline-rich motifs (PR), and NPXY and YIGM motifs. The internal M83 nucleotide deletion is illustrated. Initial methionine (M) and terminal giutamine (Q) amino acids for each mRNA are indicated, yielding proteins of 928 aa (s-SHIP) and 1191 aa (SHIP). Relative location and orientation of the primers used in the RT-PCR analysis or RACE cloning are indicated below the SHIP cONA. Numbers below the primers (arrows) represent the 5″ nucieotide in the primer based on its positioni the SHIP cDNA sequence.

Results Identification and Characterization of s-SHIP mRNA

Previous analysis of SHIP expression indicated that its expression is restricted to cells of the hematopoietic system. However, while evaluating other cell types for expression of the SHIP gene, it was found that ES cells express SHIP mRNA. The inventors detected the presence of SHIP mRNA in polyA+RNA isolated from ES cells (TL1) using several different RT-PCR assays that amplify distinct regions of SHIP mRNA (FIG. 1A,C). However, primers that amplify the 5″ portion of SHIP mRNA that encodes its SH2 domain failed to amplify the SHIP mRNA species expressed in ES cells, but they yielded the expected product when amplifying mRNA from B-lymphocyte cell lines (70Z/3, WEHI-23I). Furthermore, we found that an ES cell line (E10) with both SHIP alleles mutated by the insertion of a GFP transgene into exon 1 (J. Wang et al, unpublished data, September 1997) retains expression of SHIP mRNA as detected by several independent RT-PCR assays for SHIP expression (FIG.1A,C). These results indicate that ES cells express a SHIP transcript, which the inventors designate s-SHIP, that differs at its 5″ end from the SHIP mRNA previously described in differentiated hematopoietic cells.

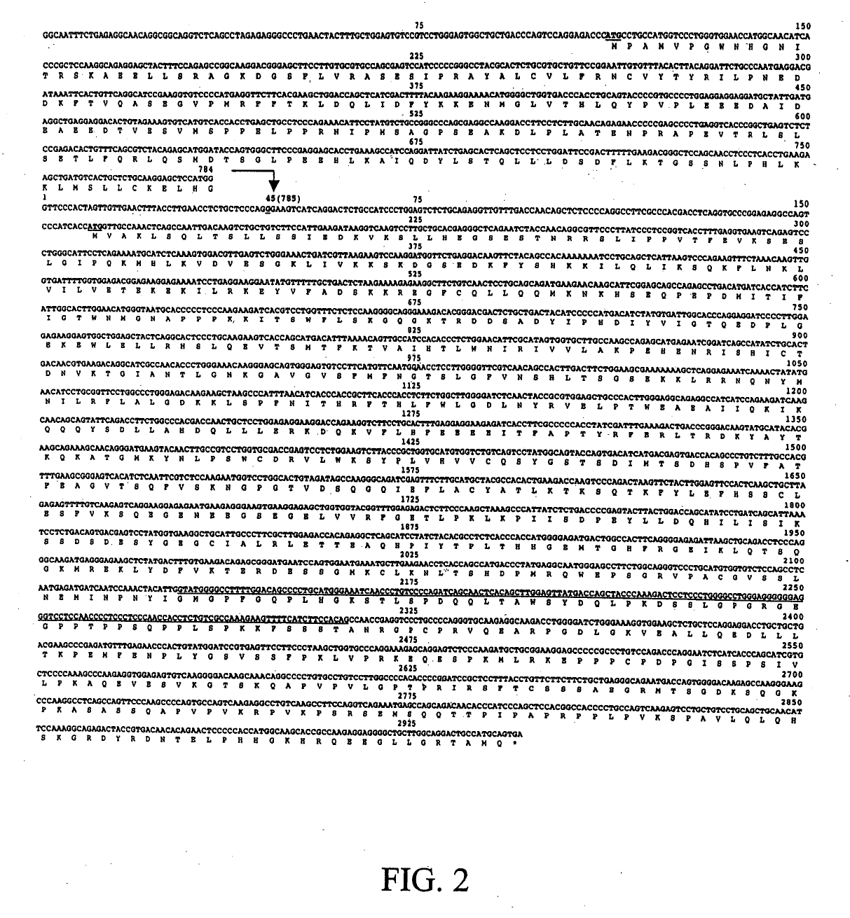

To confirm the presence of a SHIP mRNA species with a different 5″ end in ES cells and to determine its sequence, the inventors performed 5″ Smart Race cDNA cloning using antisense primers complementary to the SHIP cDNA sequence. The inventors used primers from regions of SHIP that analysis in FIG. 1A indicated were also present in s-SHIP mRNA. Two separate 5″ RACE reactions with primers ASHIPI998 and ASHIPI098 yielded a single product of either 1,3 kb (FIG. 1B) or 0.4 kb (data not shown), respectively. Sequence analysis of these 5″ RACE products showed that the SHIP mRNA present in ES cells begins with a 44-nucleotide region not previously found in any murine SHIP cDNA identified to date (FIG. 2). The inventors designate this novel 44-nucleotide region the stem-SHIP region (SSR). The complete sequence of these 5″ RACE products showed that the SSR forms the 5″ end of the s-SHIP mRNA and is fused with the previously identified murine SHIP cDNA sequence beginning at nucleotide 785 and is identical up to nucleotide 1998 (GenBank accession number U52044).

To determine the sequence of the remainder of the s-SHIP transcript present in ES cells, the inventors performed 2 separate 3″ RACE reactions (FIG. 1B) with sense strand specific primers (SHIPI97O and SH1P2766). Cloning and sequencing of these 3″ RACE products indicated that s-SHIP mRNA is identical to SHIP mRNA from nucleotide 1970 through the end of the SHIP cDNA. To further confirm the sequence of the s-SHIP cDNA, the inventors obtained full-length s-SHIP cDNA by RT-PCR. Turning to FIG. 2, sequence analysis of the full-length s-SHIP cDNA confirmed that s-SHIP mRNA lacks the SH2 domain but has the 44-nucleotide SSR fused to nucleotide 785 of SHIP. However, like its hematopoietic counter part, s-SHIP mRNA encodes an inositol 5″-phosphatase domain and several protein interaction motifs, including 2 NPXY motifs, a YIGM motif, and 4 proline-rich motifs (FIGS. 1C, 2). As Lucas and Rohrschneider found for SHIP mRNA in hematopoietic cells, s-SHIP mRNA is alternatively spliced to generate a form that lacks a 183-nucleotide region (nucleotides 2129-2311 of s-SHIP [accession number AF184912] corresponding to nucleotides 2869-3051 of SHIP). Forms of s-SHIP mRNA that contain or lack this 183-nucleotide region are present in ES cells, indicated by the RT-PCR reaction, with primers that span this expression arises from an internal promoter in the SHIP gene region (1970+3164 in FIG. 1A). Sequence analysis of these products confirmed that the alternative splice event that results in the Δ83 s-SHIP mRNA (accession number AF1 84913) is identical to that described by Lucas and Rohrschneider for SHIP.

FIG. 2 indicates the nucleotide sequence of s-SHIP cDNA and the predicted amino acid sequence of its major open reading frame (ORF). The first 784 nucleotides of the SHIP cDNA are provided for comparison above the bold arrow, s-SHIP cDNA sequence is indicated below the bold arrow, starting with the numeral 1 and continuing through nucleotide 2946. Remaining a-SHIP cDNA sequence (nucleotides 2947-4125) is not shown because of size constraints, but it is identical to nucleotides 3687 to 4865 of the SHIP cDNA (data not shown). The number in parentheses (785) at nucleotide 45 of the s-SHIP cDNA indicates the nucleotide in the SHIP cDNA, where identity between the s-SHIP and SHIP cDNAs begins. ATG initiator codons for the SHIP and s-SHIP major ORFs are indicated in bold and underlined, and the termination codon shared by both isoforms is indicated by an asterisk. SH2 (dotted box), SSR (solid box), and Inositol 5 (dashed box) sequences are shown. The nucleotide sequence that encodes the Δ183 nucleotide deletion is underlined. NPXY and proilne-rich motifs are shaded black and gray, respectively.

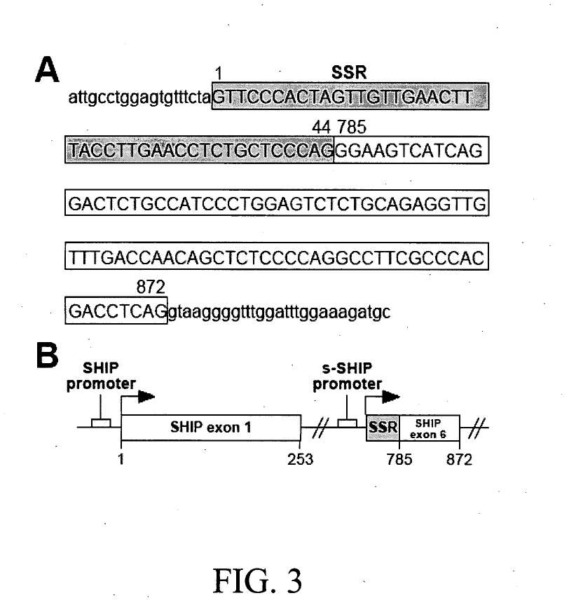

FIG. 3 shows the organization of the first exon of s-SHIP. (A) Genomic sequence of the first exon of s-SHIP and adjacent intronic sequence. Uppercase letters represent the nucleotides in the first axon of s-SHIP, and lowercase letters represent the intronic sequence immediately flanking this axon. The 44 nucleotides enclosed in the shaded box indicate the SSR, and the 88 nucleotides in the clear box represent those found in both the s-SHIP and the 145-kd SHIP cDNAs, Numbers 785 and 872 indicate the position of each corresponding nucleotide in the murine 145-kd SHIP cDNA sequence (GenBanic accession number U52044). (B) Schematic representation of the orientation of the predicted promoter regions and first axons of SHIP and s-SHIP relative to each other in the SHIP locus. Arrows indicate the transcriptional start points. The s-SHIP first axon consists of the 44-nucleotide SSR and the adjacent 88-nucleotide SHIP axon 6. Nucleotide numbering refers to the 145-kd SHIP cONA sequence (accession number U52044).

Genomic Location of the s-SHIP First Exon Indicates its Expression Arises From an Internal Promoter in the SHIP Gene

To determine whether the 44-nucleotide SSR at the 5″ end of the s-SHIP mRNA represents an independent exon or is a part of an existing exon in SHIP, the inventors isolated and sequenced the genomic DNA that spans this region. Sequence analysis of the genomic DNA revealed that the 44-nucleotide SSR is directly adjacent to a sequence that serves as exon 6 (nucleotides 785-872) in the SHIP isoform expressed in lineage-committed hematopoletic cells (FIG. 3). Thus, the 44-nucleotide SSR is not a discrete exon but is part of a larger 132-nucleotide region that serves as exon 1 in s-SHIP.

Furthermore, because the 132-nucleotide s-SHIP exon 1 is located at an internal site in the SHIP gene, s-SHIP expression must arise from an internal promoter that is 3″ to the SH2-encoding exons and 5″ to the exons that encode the inositol 5″-phosphatase domain (FIG. 3B). The location of s-SHIP exon 1 requires that its transcription initiate internally in the SHIP genomic locus in a region that serves as an intron for the SHIP mRNA expressed in differentiated hematopoietic cells.

Embryonic and Hematopoietic Stem Cells Express s-SHIP mRNA

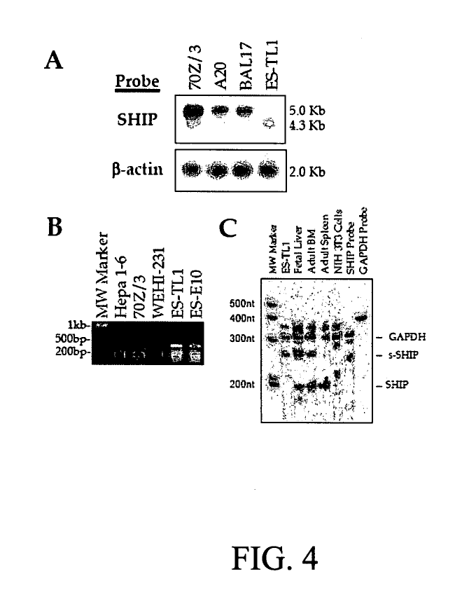

RT-PCR analysis indicated that s-SHIP is the only SHIP transcript expressed in ES cells (FIG. 1). However, to determine whether s-SHIP is also expressed in the hematopoietic system along with its SH2-encoding counterpart, the inventors analyzed total polyA+RNA isolated from ES cells and lineage-committed hematopoietic cell lines by Northern blot. This analysis showed that B-lymphocyte cell lines (70Z/3, A20, BAL17) express only the approximately 5 kb SHIP mRNA, whereas the ES cell line TLI expresses only the smaller, approximately 4.3 kb s-SHIP mRNA (FIG. 4A). In addition, an RT-PCR assay that is specific for s-SHIP expression showed that ES cells (TL1, E10) express s-SHIP, whereas B-lymphocyte cell lines (70Z/3, WEHI-23 I) do not (FIG. 4B). These results demonstrate that s-SHIP expression is silenced in differentiated hematopoietic cells.

FIG. 4. Analysis of s-SHIP and SHIP mRNA expression in ES cells and hematopoiesis. (A) Northern Blot analysis of SHIP mRNA species expressed by ES cells (TL1) and lineage-committed hematopoietic cell lines (70ZJ3, A20, BAL17). A full-length SHIP cDNA clone was used to probe the filter. After initial hybridization with the SHIP probe, the filter was stripped and reprobed for β-actin to confirm that comparable amounts of RNA were loaded in each lane. (B) RT-PCR analysis of s-SHIP expression in ES cells (TL 1, E10) and lineage-committed hematopoietic cell lines (70Z/3, WEHI-231). The primer pair ESHIP23 and ASHIPIO98 selectively amplifies s-SHIP mRNA and not SHIP mRNA. Analysis of the hepatoma cell line, Hepa 1-6, serves as a negative control for SHIP. (C) RPA analysis of s-SHIP and SHIP expression in ES cells and hematopoietic development. After annealing and RNase digestion, the s-SHIP/SHIP probe is protected for 247 nucleotides by s-SHIP mRNA and for 203 nucleotides by SHIP mRNA. Thus, a single probe is used to detect the relative abundance of the s-SHIP and SHIP mRNAs. Each RNA sample was incubated with a GAPDH probe as an internal control. Analysis of NIH3T3 cell RNA Is provided as a negative control for both SHIP and s-SHIP mRNA expression.

To further assess the relative ratio of s-SHIP to SHIP mRNA expressed in different cell types and tissues, the inventors developed an RPA that simultaneously detects both s-SHIP and SHIP mRNAs (FIG. 4C). RPA analysis indicates that s-SHIP is expressed to the exclusion of SHIP in ES cells and that SHIP mRNA is the predominant form in adult spleen. However, in FL and ABM tissues that contain significant numbers of pluripotent HSC the inventors find coexpression of s-SHIP and SHIP mRNA. Interestingly, FL has significantly more s-SHIP than SHIP mRNA, whereas ABM has slightly less s-SHIP than SHIP mRNA. These results suggest that the relative abundance of s-SHIP mRNA in a tissue may correlate to the relative abundance of HSC activity in the tissue. For instance, FL is substantially enriched for primitive HSC activity relative to ABM, whereas the spleen is a poor source of HSC activity.

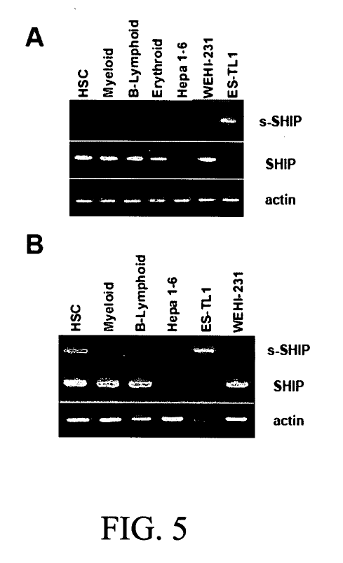

Focusing now on FIG. 5, HSCs express s-SHIP mRNA. HSCs and lineage-committed cells were sorted from ASM or day 14.5 FL and analyzed by nested RT-PCR assay for the expression of s-SHIP, SHIP, and β-actin mRNA. In both ABM and FL, s-SHIP mRNA was detected In HSCs (Sca-1+c-kit+Lin−) but not in the myeloid (Gr-1+/MAC-1+), B-lymphoid (B220+), or erythroid (Ter119+) lineages. Hepa 1-6 cells were sorted as the negative control for both s-SHIP and SHIP mRNA expression, and ES-TL1 and WEHI-231 cells were sorted as positive controls for s-SHIP and SHIP mRNA expression, respectively.

To directly assess the possibility that s-SHIP is expressed in HSC, the inventors developed a nested RT-PCR procedure to detect the expression of s-SHIP and SHIP mRNAs. To define the s-SHIP expression pattern in vivo, the inventors sorted HSC (Sca-1+cKit+Lin−) as well as cells of the B-lymphoid (B220+), myeloid (Gr-1+Mac-1), and erythroid (Ter119 lineages from day 14.5 FL or ABM. Fifty cells of each population were deposited into single wells of a 96-well PCR plate containing lysis buffer and dNTPs. RNA was reverse transcribed by adding multiple primer pairs for s-SHIP, SHIP, and β-actin together with M-MLV reverse transcriptase. Nested RT-PCR reactions were performed (see “Materials and methods”). Representative RT-PCR results from multiple independent sorts of ABM and FL are shown in FIG. 5. The inventors detected s-SHIP mRNA in Sca-1+cKit+Lin− HSC from both ABM and FL, but not in cells of the B-lymphoid, myeloid, or erythroid lineages. RT-PCR analysis of 50 sorted ES-TL1, WEHI-23 1, and Hepa 1-6 cells served as s-SHIP, SHIP, and negative controls. These results provide further support for the hypothesis that s-SHIP expression is restricted to primitive stem cell populations. The inventors also detected SHIP expression in HSC; thus, it remains to be determined whether the 2 isoforms are coexpressed in all HSC or whether they are discordantly expressed in distinct subsets of HSC.

Embryonic Stem Cells Express a Lower Molecular Weight Isoform of SHIP, Consistent with the Predicted Open-Reading Frame in the s-SHIP cDNA

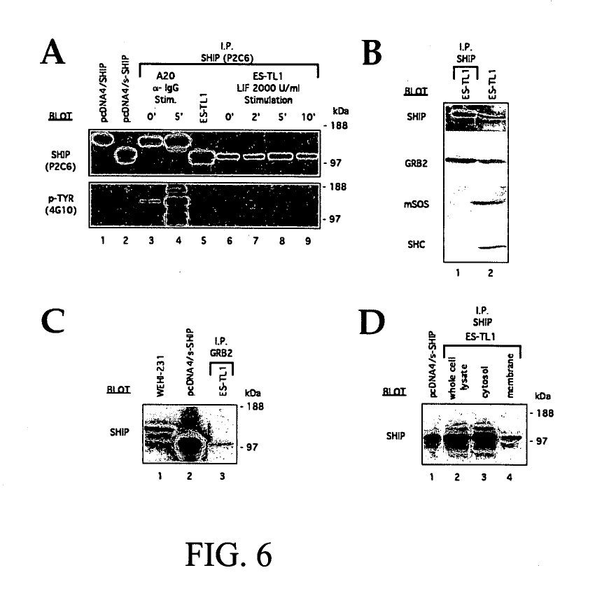

FIG. 6 illustrates how ES cells express the 8-SHIP protein isoform that associates with the Grb2 adapter protein. (A) Immunoprecipitation and immunoblot detection of s-SHIP in ES cell lysates. Lysate from ES-TL1 cells cultured in LIF was immunoprecipitated with the P2C6 anti-SHIP monoclonal antibody, separated on gels, transferred to membranes, and probed with P2C6, revealing 104-kd and 97-kd proteins (lane 5). No tyrosine phosphorylation of these proteins was detected when they were probed with the 4G10 anti-phosphotyrosine antibody. For comparison, lysates from 293T cells transfected with SHIP cDNA (lane 1) and s-SHIP cDNA (lane 2) were included in the blots. To further assess the tyrosine phosphorylation status of s-SHIP, timed LIF stimulation studies were performed. ES-TL1 cells incubated for 5 hours without LIF were stimulated with 2000 U/mL LIF for 0, 2, 5, or 10 minutes and were rapidly lysed. Equal amounts of total protein were immunoprecipitated with the P2C6 antibody and probed separately with the P2C6 and 4G10 antibodies (lanes 6-9). No tyrosine phosphorylation of s-SHIP was detected at any time point with the 4G10 antibody. For comparison, A20 8-lymphoid cells were stimulated with antiIgG antibody for 0 or 5 minutes, lysed, immunoprecipitated with P2C6, and probed with P2C6 and 4G10 (lanes 3, 4), showing prominent tyrosine phosphorylation of SHIP at 5 minutes. Molecular mass standards are indicated on the right. (B) s-SHIP associates with Grb2 but not Shc in ES cells. ES-TL1 cell lysates were prepared and immunoprecipitated with the P2C8 anti-SHIP monoclonal antibody. Resolved immunoprecipitates were then blotted with antibodies specific for SHIP, Grb2, mSosI, or Shc (lane 1). Whole cell lysates from ES-TL1 cells were also Included to confirm the expression of these proteins in ES cells (lane 2). (C) Grb2 associates with s-SHIP in ES cells. ES-TLI cell lysate was prepared and immunoprecipitated with an antiGrb2 polyclonal antibody. Resolved immunoprecipitate was then blotted with the P2C6 antiSHIP monoclonal antibody (lane 3). For comparison, whole cell lysatea from WEHI-231 cells (lane 1) and 293T cells transfected with s-SHIP cDNA (lane 2) were included. (D) Subcellular localization of s-SHIP protein in ES cells. Whole cell lysate, cytosol, and membrane fractions from ES-TLI cells were prepared as described. One milligram total protein from each fraction was immunoprecipitated with the P2C6 antiSHIP monoclonal antibody, and equal volumes of immunoprecipitate from each preparation were separated on gels, transferred, and blotted with the P2C6 antiSHIP monoclonal antibody (lanes 2-4). For comparison, whole cell lysate from 293T cells transfected with s-SHIP cDNA was induded (lane 1).The major predicted open-reading frame (ORF) in s-SHIP mRNA begins with a Kozak consensus ATO that is 3″ to the 44-nucleotide SSR (FIG. 2). This ATG and its associated ORF of 928 amino acids (an) is predicted to yield a translated product of 104 kd. Immunoprecipitation and blotting with the antiSHIP monoclonal antibody P2C6, a kind gift of Drs. David Lucas and Larry Rohrschneider, revealed a SHIP protein in ES cell lysates with an apparent molecular weight (MW) of approximately 104 kd, consistent with the predicted MW (FIG. 6A). The P2C6 antibody reacts with a region of SHIP from aa-866 to aa-I020 that, based on the cDNA sequence, should also be present in the s-SHIP isoform. This smaller s-SHIP isoform is not detected in the B-lymphoid cell lines A20 and WEHI-23 1 (FIG. 6A-C). In addition, the endoge nous s-SHIP protein immunoprecipitated from ES cells comigrated with the translated product of the s-SHIP cDNA when expressed after transfection of a non-ES cell line, 293T (FIG. 6A). Thus, this approximately 104-kd species represents the s-SHIP protein translated from the s-SHIP mRNA in ES cells, and not the COOH-terminal truncation product of SHIP with a similar MW identified by Damen et al. Furthermore, the inventors found that s-SHIP appeared as a doublet of approximately 104/97 kd in ES cells and in transfected 2931 cells (FIG. 6A-D). This doublet presumably represents the translation products of 2 different s-SHIP mRNAs (928-aa and 867-an [MW, 97.7 kd], respectively) generated by the alternative 183 nucleotide splice reaction identified in hematopoietic cells that also takes place in ES cells (FIG. 1A).

s-SHIP Forms a Complex with Grb2 and is Present Constitutively at the Membrane in Embryonic Stem Cells

The SHIP isoform expressed in mature hematopoietic cells is tyrosine phosphorylated on stimulation by growth factor, immune complexes, or BCR engagement. Phosphorylated SHIP is found associated with the adapter protein Shc, which facilitates SHIP recruitment to the plasma membrane, where it can then act on phosphatidylinositol substrates such as P1P3. However, the inventors found that s-SHIP is not tyrosine phosphorylated in either ES cells grown in LIF at steady state or ES cells deprived of LIF for 5 hours and subsequently stimulated with 2× LIF (FIG. 6A). Furthermore, s-SHIP in ES cells grown in LIF did not associate with Shc (FIG. 6B). Instead, immunoprecipitation of s-SHIP by the P2C6 anti-SHIP antibody coimmunoprecipitated the adapter protein Grb2. Similarly, immunoprecipitation with a poly clonal antibody specific for Grb2 coimmunoprecipitated s-SHIP (FIG. 6C). These results demonstrate an in vivo association between s-SHIP and the major adapter protein Grb2 in totipotent stem cells that does not require tyrosine phosphorylation of s-SHIP. s-SHIP does not appear to be associated with either Shc or mSosI in ES cells, suggesting that it may have different preferences for partnering than the SHIP isoform (FIG. 6B). Immunoblotting of ES cell lysates confirmed that Shc and mSosI are expressed in ES cells. To further assess the potential for s-SHIP to play a signaling role in primitive stem cells, the inventors prepared cytosolic and membrane fractions of ES cells. Immunoprecipitation of s-SHIP from these subcellular fractions (FIG. 6D) demonstrates the presence of s-SHIP protein in the membrane fraction of ES cells. This membrane localization of s-SHIP and its association with a major adapter protein indicate a role for s-SHIP in signaling pathways active in primitive stem cells,s-SHIP demonstrates significant homology with the human 11O-kd signaling inositol polyphosphate 5″-phosphatase

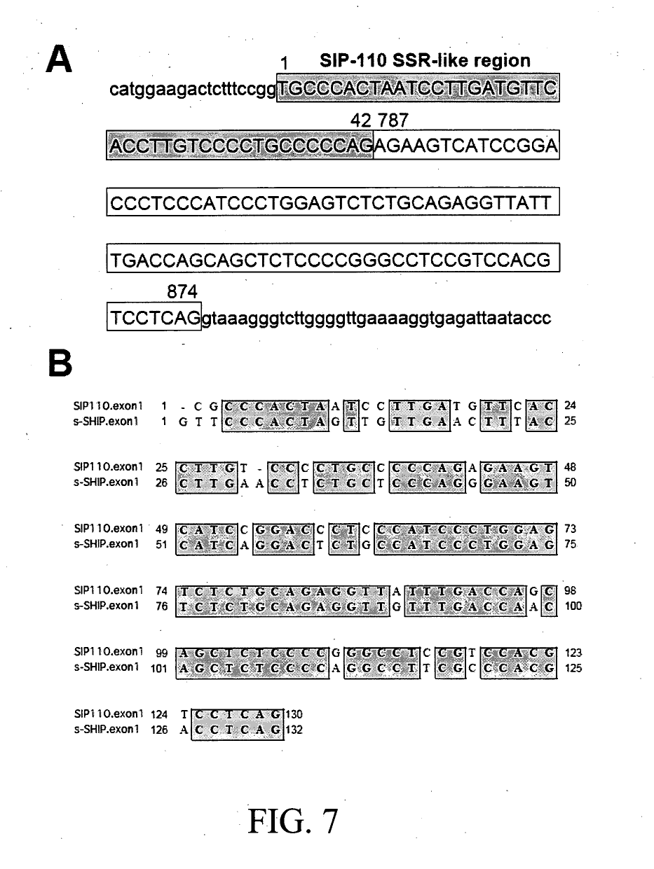

Clustal W Sequence Analysis revealed that s-SHIP and the human SHIP isoform SIP-110 (GenBank accession number U50040), described by Kavanaugh et al,” share 78% nucleotide identity. Of note, Kavanaugh et al predicted a 976-an protein (calculated MW, 109 kd) based on an ATG initiation codon at position 19 of GenBank accession number U50040. This ATG initiation site lacks strong Kozak consensus features. However, a second ATG initiation codon at position 160 displays a strong Kozak consensus sequence of CAnCATGG and would predict a translated protein of 929 aa (calculated MW, 104 kd). This shorter ORF of SIP-I10 shares 88% amino acid identity with the s-SHIP ORF. Nucleotides 43-2786 of human SIP-110 eDNA are identical to nucleotides 787-3537 of human I45-kd SHIP eDNA (GenBank accession number U50041). Reminiscent of s-SHIP nucleotides 1-42 of SIP-110 are not found in the 145-kd SHIP eDNA. Kavanaugh et aP ptup that SIP-110 is a splice variant of the 145-kd SHIP. However, the inventors hypothesized that SIP-110 may represent the human homologue of s-SHIP and may arise by alternative promoter use. To confirm this, the inventors conducted a BlastN query of the first 120 nucleotides of SIP-I10 in the Celera Human Genome-Unassembled Fragments database. The inventors found 2 unassembled and uncharacterized genomic fragments (Celera accession numbers GA_x4N24A5JORM: I 0.262 and GA_x4N24A6F9UD: 1.508) that span the first exon of SIP-110. These genomic fragments revealed that the first 42 nucleotides of SIP-110 are found in the SHIP intronic sequence and are followed directly by an 88-nucleotide sequence representing an internal SHIP exon corresponding to nucleotides 787-874 of 145-kd SHIP (FIG. 7A). Thus, analogous to s-SHIP, the first exon of SIP-110 is comprised of a 42-nucleotide SSR-like region unique to SIP-110 and an adjacent 88-nucleotide exon shared by both SIP-110 and I45-kd SHIP. The first exons of SIP-110 and s-SHIP show 82% nucleotide identity (FIG. 7B). These results establish that SIP-110 is the human homologue of s-SHIP.

Discussion

The SHIP protein is a key signaling component that participates in controlling the responses of several different hematopoietic cell types in the adult mouse, 1-3, 13, 16,25. Results provide the first evidence that a SHIP isoform plays a specific role in the biologic composition of stem cell populations, Four lines of evidence support this hypothesis: (1) Totipotent ES cells express the s-SHIP isoform exclusively, (2) s-SHIP partners preferentially with Grb2 in ES cells, (3) s-SHIP is present constitutively in the membrane fraction of ES cells, and (4) s-SHIP expression within the hematopoietic compartment is restricted to fetal and adult hematopoietic stem cells. Results also suggest the evolution of a transcription control mechanism that promotes the expression of s-SHIP in Stem cell populations.

The striking discordance between stem cells and mature hematopoietic cells in the expression of s-SHIP mRNA suggests a distinct signaling role for this SHIP isoform in stem cells. The SH2 domain of SHIP allows it to participate in signal transduction pathways in mature hematopoietic cells that involve partnering with Shc or binding to ITM motifs of receptors. The lack of an SH2 domain in s-SHIP may change its binding preferences and, therefore, the pathways with which it interacts. Consistent with this hypothesis, the inventors detected no association of s-SHIP with Shc in ES cells at steady state or after LIF stimulation. The inventors did find an association of s-SHIP with Grb2 in ES cells in the absence of tyrosine phosphorylation. Several groups have shown that SHIP can associate with Grb2 in stimulated hematopoietic cells through an interaction of the SH3 domain of Grb2 and the proline-rich motifs of SHIP. s-SHIP contains these same proline-rich motifs. In addition to SH3 domains, Grb2 contains an SH2 domain that enables its binding to other signal transduction components or receptors at the cell membrane. Grb2, through its SH2 domain, could recruit s-SHIP to tyrosine-phosphorylated receptors that are important in the biologic makeup of ES cells and HSCs. Thus, the absence of an SH2 domain may not preclude s-SHIP from acting at the cell membrane, but it may alter how it is recruited to the cell membrane and which signaling pathways it impacts.

Membrane recruitment of s-SHIP would enable its inositol 5″-phosphatase domain to access phosphatidylinositols such as PIP3 and to influence proliferation, differentiation, or apoptosis of stem cell populations. Because the association of s-SHIP with Grb2 does not appear to require tyrosine phosphorylation, this complex is likely to be present in quiescent stem cell populations. This preformed complex could rapidly associate with receptors that become tyrosine phosphorylated after basal level growth factor stimulation, enabling s-SHIP to prevent the accumulation of PIP3 to significant levels. In this way, s-SHIP could participate in the process of maintaining a stem cell population in a quiescent state. Furthermore, analogous to the proposal that SHIP interferes with the mSosI/Shc/Grb2 complex of the MAPK pathway in mature cells, one can envision a competition in stem cells between s-SHIP and mSosI for Grb2. The outcome of this competition may influence the decision to remain quiescent or to activate the Ras/MAPK pathway that triggers proliferation and differentiation.

FIG. 7 depicts the organization of the first axon of SIP-110, the human homolog of s-SHIP. (A) Genomic sequence of the SIP-110 first exon and adjacent intronic sequence as compiled from the Celera Human Genome-Unassembled Fragments database (Celera accession numbers GA_x4N24A5.JORM: 1 0.262 and GA_x4N24A6F9UD:1.508). Uppercase letters represent the nucleotides in the SIP-110 first exon, and lowercase letters represent the intronic sequence immediately flanking this exon. The 42 nucleotides enclosed in the shaded box indicate the SSR-like region, and the 88 nucleotides in the clear box represent those nucleotides found in both the SIP-110 and the human 145-kd SHIP cDNAs. Based on the genomic fragment sequences, position 1 is listed as “T” rather than as the “C” assigned in the SIP-110 cDNA (GenBsnk accession number U50040). Numbers 787 and 874 indicate the position of the corresponding nucleotide in the human 145-kd SHIP cDNA sequence (Gen Bank accession number U50041). (B) Clustal W alignment of the first exon sequences of human SIP-110 and murine s-SHIP. Matching nucleotides are shaded gray. Overall, the first axons of SIP-110 and s-SHIP show 82% nucleotide identity.

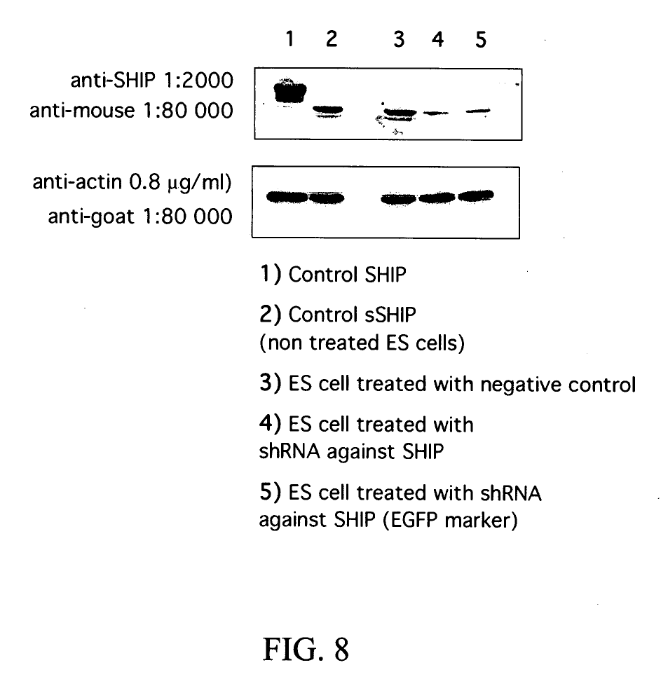

FIG. 8 demonstrates the decrease in s-SHIP expression after electroporation of embryonic stem cells with anti-SHIP shRNA. The electroporation conditions where as follows: 10 μg of protein were loaded into a 4-12% Bis-Tris gel (Invitrogen), 250V, 500 μF, 15 μg of plasmid, and sample was left to rest on ice, in cold PBS, for 30 minutes before being placed in a warm media.

The identification of a distinct SHIP isoform expressed from an internal site within the SHIP gene suggests the possibility that the recently reported SHIP-null mice may not be absolute nulls for expression from the SHIP locus. 1-3,25,48-50 The mutation strategy used by both groups involved the insertional mutation of the first exon that encodes a portion of the SH2 domain at the amino terminus of the SHIP isoform. This insertional mutation strategy clearly leads to the ablation of the SHIP isoform in mature hematopoietic cells. However, one would not predict that expression of the s-SHIP isoform in the embryo or in HSCs would be affected given that the s-SHIP first exon consists of the terminal 44 nucleotides of intron V, together with exon 6 of SHIP. This region is approximately 27 kb downstream of the 145-kd SHIP promoter. Nevertheless, ablation of SHIP expression by this strategy may lead to the dysregulation of s-SHIP expression by an unknown mechanism. In preliminary experiments, the inventors were unable to detect s-SHIP protein expression from total bone marrow of adult C57BL/6 mice in which the first exon of SHIP was insertionally mutated (data not shown). If it can be determined that s-SHIP expression during embryogenesis and hematopoietic development is intact in these SHIP-null strains, one might predict a novel phenotype in mice designed to be null for both s-SHIP and SHIP.

Kavanaugh et al” cloned and characterized the human SHIP isoform, SIP-110 that lacks an SH2 domain. They cloned SIP-110 cDNA (GenBank accession number U50040) from a human placenta cDNA expression library based on its affinity for a Grb2 SH3 domain in the absence of tyrosine phosphorylation. The overall structure of SIP-110 was similar to s-SHIP. However, SIP-110 was described as a 976-aa protein with an estimated molecular weight of 110 kd. Although the origin of the first 42 nucleotides of SIP-110 within the human SHIP gene was unknown, it was proposed that SIP-110 results from an alternative splice event. Analysis of the structure and the genomic location of the first exon of SIP-110 confirms that SIP-110 is the human homologue of s-SHIP. Furthermore, the structure and internal location of the SIP-110 first exon within the SHIP gene indicates that SIP-110 arises through alternative promoter use rather than as a splice variant, as was previously proposed. Although there are 2 potential ORFs for SIP-110 that predict proteins of 976 as and 929 as, the distal ATG initiation codon has Kozak consensus features that may favor translation of the 929-aa, 1 04-kd protein, which more closely resembles s-SHIP. Results demonstrate that s-SHIP is expressed in murine HSCs. Because cord blood within placenta is known to be enriched for HSCs, the inventors hypothesize that the SIP-I 10 cDNA isolated by Kavanaugh et al” may have originated from cord blood HSC cDNA present in the placenta library.

In vitro studies with recombinant SIP-110 confirmed that it can bind Grb2, but its inositol 5″-phosphatase activity is unaffected by Grb2 binding.” Furthermore, SIP-110 did not associate with Shc. These results can be extrapolated to s-SHIP, and they suggest that the purpose of Grb2 binding is not to alter s-SHIP enzymatic activity but rather to direct the subcellular localization of this activity.

Geier et al similarly found a I 0O-kd isoform of SHIP in both human and murine bone marrow using a polyclonal antibody to an amino acid sequence of marine SHIP (as 670 to as 868) that is also present in s-SHIP. Interestingly, they also found that a 1 00-kd isoform was the most prominent species in human peripheral blood mononuclear cells. Examination of the human ML-1 myeloblastic leukemia cell line revealed the presence of a 100-kd SHIP isoform and a 105-kd isoform and the absence of higher MW isoforms. When these ML-I myeloblasts were induced to differentiate into monocytes in the presence of 12-O-tetradecanoylphorbol-13-acetate, higher MW isoforms (175 kd, 145 kd, and 130 kd) appeared, whereas the lower MW isoforms (105 kd, 100 kd) were no longer detectable. These results with ML-1 cells indicate that as human myeloid cells differentiate, human SHIP expression changes from the lower MW 105-kd and 100-kd isoforms to the higher MW isoforms. This is analogous to what the inventors found with s-SHIP and SHIP isoforms in murine hematopoietic cell development, and it suggests that the lower MW human isoforms characterized by Geier et al represent the SIP-110 identified by Kavanaugh et al.” However, it is unclear why in their studies human peripheral blood displayed only the lower MW 100-kd isoform. Wolf et al describe an additional 110-kd SHIP isoform, SHIP, which is the product of an out-of-frame splice event with a deletion of 167 nucleotides in the C-terminal region. This clearly represents a different isoform than s-SHIP. It remains to be determined which SHIP-related proteins, among these several isoforms encoding 100 kd to 110 kd, predominate during different stages of hematopoietic development.

Recently, Krause et al elegantly demonstrated that a single murine adult bone marrow stem cell can repopulate hematopoietic cells and a diverse array of nonhematopoietic epithelial tissues, including lung, liver, gastrointestinal tract, and skin. Given that s-SHIP is expressed in totipotent ES cells and at least a subset of bone marrow HSCs, this study raises the possibility that s-SHIP expression may serve to further characterize and delineate the pluripotent bone marrow stem cell capable of multi-organ engraftment that Krause et al identified.

It will be seen that the objects set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be the to fall therebetween. Now that the invention has been described,

We claim:

1. A method for identifying the presence of stem cells in a cell sample, comprising detecting the presence of cells expressing s-SHIP RNA within the cell sample.

2. The method of claim 1, wherein the presence of stem cells in the sample is indicated by the presence of s-SHIP enzymatic activity.

3. The method of claim 1, wherein said detecting comprises adding anti-SHIP monoclonal antibody P2C6 or P1C1 to the sample under conditions, and for a time, suitable for antibody binding; and detecting the presence of the antibodies bound to the cells in the sample, wherein the presence of stem cells in the sample is indicated by cells that express the s-SHIP isoform.

4. The method of claim 3, wherein the antibodies are labeled with a flourochrome.

5. The method of claim 4, wherein the detection of antibodies is by fluorescence activated cell sorting.

6. The method of claim 1, wherein the stem cells are embryonic stem cells.

7. The method of claim 1, wherein the stem cells are tissue-specific stem cells.

8. The method of claim 7, wherein then stem cells are hematopoietic stem cells.

9. The method of claim 3, wherein the stem cells are hematopoietic stem cells and the monoclonal antibody is P2C6.

10. A method for inhibiting proliferation of stem cells, comprising contacting the cells with s-SHIP thereby increasing competition with mSosI for Grb2 in the stem cells.

11. The method of claim 10, wherein the stem cells are selected from the group consisting of hematopoietic progenitors of mature blood cells, hematopoietic progenitors of lymph cells, and embryonic stem cells.

12. A method of inhibiting differentiation of stem cells, comprising contacting the stem cells with s-SHIP enzyme whereby the accumulation of the s-SHIP enzyme prevents the accumulation of PI³,⁴,⁵,P₃, or other signaling pathways and intermediates influenced by s-SHIP activity.

13. The method of claim 12, wherein the stem cells are selected from the group consisting of hematopoietic progenitors of mature blood cells and hematopoietic progenitors of lymphoid cells.

14. A recombinant nucleic acid promoter fragment isolated from an internal region of the SHIP gene where s-SHIP transcription is controlled, wherein the promoter fragment comprises a nucleotide fragment internal to the SHIP gene.

15. The recombinant nucleotide promoter fragment of claim 14, wherein the amino acid sequence is selected from the group consisting of SEQ ID NO: 4, SEQ ID NO: 5, SEQ ID NO: 6, SEQ ID NO: 7, SEQ ID NO: 8, SEQ ID NO: 9, and SEQ ID NO: 10.

16. The recombinant nucleotide promoter fragment of claim 14, wherein the fragment is found in stem cells selected from the group consisting of: murine embryonic stem cells, murine hematopoietic stem cells, human embryonic stem cells, and human hematopoietic stem cells.

17. A method for inducing proliferation of stem cells, comprising introducing anti-SHIP shRNA into the cell by electroporation.

18. The method of claim 17, wherein the stem cells are selected from the group consisting of hematopoietic progenitors of mature blood cells, hematopoietic progenitors of lymph cells, and embryonic stem cells.

19. A method for identifying stem cells in a cell sample, comprising detecting s-SHIP by immunofluorescence.

20. The method of claim 19, wherein the cell sample is a complex mixture of cells.

21. The method of claim 20, wherein the cell sample is in vivo.

22. The method of claim 20, wherein the presence of stem cells is indicated by the presence of the s-SHIP isoform.

23. A method for inducing proliferation in stem cells, comprising introducing an inhibitor of s-SHIP activity.

24. The method of claim 23, wherein the inhibitor of s-SHIP is a dominant negative mutant.

25. The method of claim 23, wherein the inhibitor of s-SHIP is shRNA.

26. The method of claim 23, wherein the stem cells are selected from the group consisting of hematopoietic progenitors of mature blood cells, hematopoietic progenitors of lymph cells, and embryonic stem cells.

27. The method of claim 23, wherein the stem cells are induced to differentiate.

28. A method for identifying stem cell-specific signaling components, comprising contacting the signaling component with s-SHIP.


SEQUENCE LISTING (18 sequences)

SEQ ID NO: 1 - protein, 4125 aa, Mus musculus
GTTCCCACTAGTTGTTGAACTTTACCTTGAACCTCTGCTCCCAGGGAAGTCATCAGGACTCTGCCATCCCTGGAGTCTCTGCAGAGGTTGTTTGACCAACAGCTCTCCCCAGGCCTTCGCCCACGACCTCAGGTGCCCGGAGAGGCCAGTCCCATCACCATGGTTGCCAAACTCAGCCAATTGACAAGTCTGCTGTCTTCCATTGAAGATAAGGTCAAGTCCTTGCTGCACGAGGGCTCAGAATCTACCAACAGGCGTTCCCTTATCCCTCCGGTCACCTTTGAGGTGAAGTCAGAGTCCCTGGGCATTCCTCAGAAAATGCATCTCAAAGTGGACGTTGAGTCTGGGAAACTGATCGTTAAGAAGTCCAAGGATGGTTCTGAGGACAAGTTCTACAGCCACAAAAAAATCCTGCAGCTCATTAAGTCCCAGAAGTTTCTAAACAAGTTGGTGATTTTGGTGGAGACGGAGAAGGAGAAAATCCTGAGGAAGGAATATGTTTTTGCTGACTCTAAGAAAAGAGAAGGCTTCTGTCAACTCCTGCAGCAGATGAAGAACAAGCATTCGGAGCAGCCAGAGCCTGACATGATCACCATCTTCATTGGCACTTGGAACATGGGTAATGCACCCCCTCCCAAGAAGATCACGTCCTGGTTTCTCTCCAAGGGGCAGGGAAAGACACGGGACGACTCTGCTGACTACATCCCCCATGACATCTATGTGATTGGCACCCAGGAGGATCCCCTTGGAGAGAAGGAGTGGCTGGAGCTACTCAGGCACTCCCTGCAAGAAGTCACCAGCATGACATTTAAAACAGTTGCCATCCACACCCTCTGGAACATTCGCATAGTGGTGCTTGCCAAGCCAGAGCATGAGAATCGGATCAGCCATATCTGCACTGACAACGTGAAGACAGGCATCGCCAACACCCTGGGAAACAAGGGAGCAGTGGGAGTGTCCTTCATGTTCAATGGAACCTCCTTGGGGTTCGTCAACAGCCACTTGACTTCTGGAAGCGAAAAAAAGCTCAGGAGAAATCAAAACTATATGAACATCCTGCGGTTCCTGGCCCTGGGAGACAAGAAGCTAAGCCCATTTAACATCACCCACCGCTTCACCCACCTCTTCTGGCTTGGGGATCTCAACTACCGCGTGGAGCTGCCCACTTGGGAGGCAGAGGCCATCATCCAGAAGATCAAGCAACAGCAGTATTCAGACCTTCTGGCCCACGACCAACTGCTCCTGGAGAGGAAGGACCAGAAGGTCTTCCTGCACTTTGAGGAGGAAGAGATCACCTTCGCCCCCACCTATCGATTTGAAAGACTGACCCGGGACAAGTATGCATACACGAAGCAGAAAGCAACAGGGATGAAGTACAACTTGCCGTCCTGGTGCGACCGAGTCCTCTGGAAGTCTTACCCGCTGGTGCATGTGGTCTGTCAGTCCTATGGCAGTACCAGTGACATCATGACGAGTGACCACAGCCCTGTCTTTGCCACGTTTGAAGCGGGAGTCACATCTCAATTCGTCTCCAAGAATGGTCCTGGCACTGTAGATAGCCAAGGGCAGATCGAGTTTCTTGCATGCTACGCCACACTGAAGACCAAGTCCCAGACTAAGTTCTACTTGGAGTTCCACTCAAGCTGCTTAGAGAGTTTTGTCAAGAGTCAGGAAGGAGAGAATGAAGAGGGAAGTGAAGGAGAGCTGGTGGTACGGTTTGGAGAGACTCTTCCCAAGCTAAAGCCCATTATCTCTGACCCCGAGTACTTACTGGACCAGCATATCCTGATCAGCATTAAATCCTCTGACAGTGACGAGTCCTATGGTGAAGGCTGCATTGCCCTTCGCTTGGAGACCACAGAGGCTCAGCATCCTATCTACACGCCTCTCACCCACCATGGGGAGATGACTGGCCACTTCAGGGGAGAGATTAAGCTGCAGACCTCCCAGGGCAAGATGAGGGAGAAGCTCTATGACTTTGTGAAGACAGAGCGGGATGAATCCAGTGGAATGAAATGCTTGAAGAACCTCACCAGCCATGACCCTATGAGGCAATGGGAGCCTTCTGGCAGGGTCCCTGCATGTGGTGTCTCCAGCCTCAATGAGATGATCAATCCAAACTACATTGGTATGGGGCCTTTTGGACAGCCCCTGCATGGGAAATCAACCCTGTCCCCAGATCAGCAACTCACAGCTTGGAGTTATGACCAGCTACCCAAAGACTCCTCCCTGGGGCCTGGGAGGGGGGAGGGTCCTCCAACCCCTCCCTCCCAACCACCTCTGTCGCCAAAGAAGTTTTCATCTTCCACAGCCAACCGAGGTCCCTGCCCCAGGGTGCAAGAGGCAAGACCTGGGGATCTGGGAAAGGTGGAAGCTCTGCTCCAGGAGGACCTGCTGCTGACGAAGCCCGAGATGTTTGAGAACCCACTGTATGGATCCGTGAGTTCCTTCCCTAAGCTGGTGCCCAGGAAAGAGCAGGAGTCTCCCAAGATGCTGCGGAAGGAGCCCCCGCCCTGTCCAGACCCAGGAATCTCATCACCCAGCATCGTGCTCCCCAAAGCCCAAGAGGTGGAGAGTGTCAAGGGGACAAGCAAACAGGCCCCTGTGCCTGTCCTTGGCCCCACACCCCGGATCCGCTCCTTTACCTGTTCTTCTTCTGCTGAGGGCAGAATGACCAGTGGGGACAAGAGCCAAGGGAAGCCCAAGGCCTCAGCCAGTTCCCAAGCCCCAGTGCCAGTCAAGAGGCCTGTCAAGCCTTCCAGGTCAGAAATGAGCCAGCAGACAACACCCATCCCAGCTCCACGGCCACCCCTGCCAGTCAAGAGTCCTGCTGTCCTGCAGCTGCAACATTCCAAAGGCAGAGACTACCGTGACAACACAGAACTCCCCCACCATGGCAAGCACCGCCAAGAGGAGGGGCTGCTTGGCAGGACTGCCATGCAGTGAGCTGCTGGTGATCGGAGCCTGGAGGAACAGCACAAAGCAGACCTGCGCCTCTCTCAGGATGCCTCTCTCAGGATGCCTCTTGGAGGACCTCCTGCTAGCTCTTCTTGCCTAGCTTCAAGTCCCAGGCTGTGTATTTTTTTTCAGGAAACGGCCTCACTTCTCTGTGGTCCAAGAAGTGTGCTGCTGGCTGCCACACTGTGCGGCAGATGCTAAAGCTGGATGACAAACGCACGCCATACAGACAGCAGACAGCGGCACTGGGTCTCAGAACTTGGATTCCTGGGCCTTCTTCCAGTCGCCGTTTTAAAGAAAGGAACTAACGGAGCTGCTCATCCGAGGGTGAAGATATAAATAATAATATTATTAATAATAATAACAGTCAGGTGCCATGTGCTGTGTTAAGTGCTTTATGAACATTTGTCGGGCTGGCCTCCAGTGCTGAGGTGCCAGTCAGCCTGAACCCTATGCCCAGGCCCACTAATCCCAAATGGTGGGTCCTGAGATGTTTTTAAAAAGCATTAAAGAAAACCATCGGTCTCTTAGAGCTAACCGGCCGGGCTCTACTGCAGGGACCCGAACAGTCTGCATGGCTAAGTGGCACAAGGAGCCTGGCCCTGTCCAGCTTCAGAGATCCAAGCTGCTTTTTGCTGGGGTTCTGTCACAGGCCTGATCCTCTTGGTTTTTATGGGGTTTCAAGTCTGCCAGAGTCAGAAATCAGCTCTAACTCGCCAGTGAAGAGATCTGGCCTTAACTTAAGCCAGCCACGTCAGGCCCCTGCTGAGCCTATGGACCAATAAATACTCCCCGTGCCACTGGAGGTGGGCAGCTATCACCATACCCTGAGTTGGGCCAAGCCCACCCCACCCCTACCCTGCAACATTTCTGATGTWCTGAGGAAGAGTCTCCACCATAGTCCCCAAGGGCTGAGTTCTCCAGCCTGCTATCAGGGAAGGTGAGCATTGGTCCCAGGCTCTCAAAATAGTGCAGCCTCTTCTTCCCAAGCTCTGGGGTGCACCCTGTGTCCTTGGTTACCAGGAGACTAGGGTTGTGATATCTTTTCTTGTCTTGCTTTTTGATATATCAGGATTAATGTAGGAAACCAGACCTAGATTATTCAGGAGAGTAGGTATATCCCCTGTGTTTCCCAGTCTGAGTGACCAATAAAATTGTGCCTTTCTA

SEQ ID NO: 2 - protein, 3942 aa, Mus musculus
GTTCCCACTAGTTGTTGAACTTTACCTTGAACCTCTGCTCCCAGGGAAGTCATCAGGACTCTGCCATCCCTGGAGTCTCTGCAGAGGTTGTTTGACCAACAGCTCTCCCCAGGCCTTCGCCCACGACCTCAGGTGCCCGGAGAGGCCAGTCCCATCACCATGGTTGCCAAACTCAGCCAATTGACAAGTCTGCTGTCTTCCATTGAAGATAAGGTCAAGTCCTTGCTGCACGAGGGCTCAGAATCTACCAACAGGCGTTCCCTTATCCCTCCGGTCACCTTTGAGGTGAAGTCAGAGTCCCTGGGCATTCCTCAGAAAATGCATCTCAAAGTGGACGTTGAGTCTGGGAAACTGATCGTTAAGAAGTCCAAGGATGGTTCTGAGGACAAGTTCTACAGCCACAAAAAAATCCTGCAGCTCATTAAGTCCCAGAAGTTTCTAAACAAGTTGGTGATTTTGGTGGAGACGGAGAAGGAGAAAATCCTGAGGAAGGAATATGTTTTTGCTGACTCTAAGAAAAGAGAAGGCTTCTGTCAACTCCTGCAGCAGATGAAGAACAAGCATTCGGAGCAGCCAGAGCCTGACATGATCACCATCTTCATTGGCACTTGGAACATGGGTAATGCACCCCCTCCCAAGAAGATCACGTCCTGGTTTCTCTCCAAGGGGCAGGGAAAGACACGGGACGACTCTGCTGACTACATCCCCCATGACATCTATGTGATTGGCACCCAGGAGGATCCCCTTGGAGAGAAGGAGTGGCTGGAGCTACTCAGGCACTCCCTGCAAGAAGTCACCAGCATGACATTTAAAACAGTTGCCATCCACACCCTCTGGAACATTCGCATAGTGGTGCTTGCCAAGCCAGAGCATGAGAATCGGATCAGCCATATCTGCACTGACAACGTGAAGACAGGCATCGCCAACACCCTGGGAAACAAGGGAGCAGTGGGAGTGTCCTTCATGTTCAATGGAACCTCCTTGGGGTTCGTCAACAGCCACTTGACTTCTGGAAGCGAAAAAAAGCTCAGGAGAAATCAAAACTATATGAACATCCTGCGGTTCCTGGCCCTGGGAGACAAGAAGCTAAGCCCATTTAACATCACCCACCGCTTCACCCACCTCTTCTGGCTTGGGGATCTCAACTACCGCGTGGAGCTGCCCACTTGGGAGGCAGAGGCCATCATCCAGAAGATCAAGCAACAGCAGTATTCAGACCTTCTGGCCCACGACCAACTGCTCCTGGAGAGGAAGGACCAGAAGGTCTTCCTGCACTTTGAGGAGGAAGAGATCACCTTCGCCCCCACCTATCGATTTGAAAGACTGACCCGGGACAAGTATGCATACACGAAGCAGAAAGCAACAGGGATGAAGTACAACTTGCCGTCCTGGTGCGACCGAGTCCTCTGGAAGTCTTACCCGCTGGTGCATGTGGTCTGTCAGTCCTATGGCAGTACCAGTGACATCATGACGAGTGACCACAGCCCTGTCTTTGCCACGTTTGAAGCGGGAGTCACATCTCAATTCGTCTCCAAGAATGGTCCTGGCACTGTAGATAGCCAAGGGCAGATCGAGTTTCTTGCATGCTACGCCACACTGAAGACCAAGTCCCAGACTAAGTTCTACTTGGAGTTCCACTCAAGCTGCTTAGAGAGTTTTGTCAAGAGTCAGGAAGGAGAGAATGAAGAGGGAAGTGAAGGAGAGCTGGTGGTACGGTTTGGAGAGACTCTTCCCAAGCTAAAGCCCATTATCTCTGACCCCGAGTACTTACTGGACCAGCATATCCTGATCAGCATTAAATCCTCTGACAGTGACGAGTCCTATGGTGAAGGCTGCATTGCCCTTCGCTTGGAGACCACAGAGGCTCAGCATCCTATCTACACGCCTCTCACCCACCATGGGGAGATGACTGGCCACTTCAGGGGAGAGATTAAGCTGCAGACCTCCCAGGGCAAGATGAGGGAGAAGCTCTATGACTTTGTGAAGACAGAGCGGGATGAATCCAGTGGAATGAAATGCTTGAAGAACCTCACCAGCCATGACCCTATGAGGCAATGGGAGCCTTCTGGCAGGGTCCCTGCATGTGGTGTCTCCAGCCTCAATGAGATGATCAATCCAAACTACATTGCCAACCGAGGTCCCTGCCCCAGGGTGCAAGAGGCAAGACCTGGGGATCTGGGAAAGGTGGAAGCTCTGCTCCAGGAGGACCTGCTGCTGACGAAGCCCGAGATGTTTGAGAACCCACTGTATGGATCCGTGAGTTCCTTCCCTAAGCTGGTGCCCAGGAAAGAGCAGGAGTCTCCCAAGATGCTGCGGAAGGAGCCCCCGCCCTGTCCAGACCCAGGAATCTCATCACCCAGCATCGTGCTCCCCAAAGCCCAAGAGGTGGAGAGTGTCAAGGGGACAAGCAAACAGGCCCCTGTGCCTGTCCTTGGCCCCACACCCCGGATCCGCTCCTTTACCTGTTCTTCTTCTGCTGAGGGCAGAATGACCAGTGGGGACAAGAGCCAAGGGAAGCCCAAGGCCTCAGCCAGTTCCCAAGCCCCAGTGCCAGTCAAGAGGCCTGTCAAGCCTTCCAGGTCAGAAATGAGCCAGCAGACAACACCCATCCCAGCTCCACGGCCACCCCTGCCAGTCAAGAGTCCTGCTGTCCTGCAGCTGCAACATTCCAAAGGCAGAGACTACCGTGACAACACAGAACTCCCCCACCATGGCAAGCACCGCCAAGAGGAGGGGCTGCTTGGCAGGACTGCCATGCAGTGAGCTGCTGGTGATCGGAGCCTGGAGGAACAGCACAAAGCAGACCTGCGCCTCTCTCAGGATGCCTCTCTCAGGATGCCTCTTGGAGGACCTCCTGCTAGCTCTTCTTGCCTAGCTTCAAGTCCCAGGCTGTGTATTTTTTTTCAGGAAACGGCCTCACTTCTCTGTGGTCCAAGAAGTGTGCTGCTGGCTGCCACACTGTGCGGCAGATGCTAAAGCTGGATGACAAACGCACGCCATACAGACAGCAGACAGCGGCACTGGGTCTCAGAACTTGGATTCCTGGGCCTTCTTCCAGTCGCCGTTTTAAAGAAAGGAACTAACGGAGCTGCTCATCCGAGGGTGAAGATATAAATAATAATATTATTAATAATAATAACAGTCAGGTGCCATGTGCTGTGTTAAGTGCTTTATGAACATTTGTCGGGCTGGCCTCCAGTGCTGAGGTGCCAGTCAGCCTGAACCCTATGCCCAGGCCCACTAATCCCAAATGGTGGGTCCTGAGATGTTTTTAAAAAGCATTAAAGAAAACCATCGGTCTCTTAGAGCTAACCGGCCGGGCTCTACTGCAGGGACCCGAACAGTCTGCATGGCTAAGTGGCACAAGGAGCCTGGCCCTGTCCAGCTTCAGAGATCCAAGCTGCTTTTTGCTGGGGTTCTGTCACAGGCCTGATCCTCTTGGTTTTTATGGGGTTTCAAGTCTGCCAGAGTCAGAAATCAGCTCTAACTCGCCAGTGAAGAGATCTGGCCTTAACTTAAGCCAGCCACGTCAGGCCCCTGCTGAGCCTATGGACCAATAAATACTCCCCGTGCCACTGGAGGTGGGCAGCTATCACCATACCCTGAGTTGGGCCAAGCCCACCCCACCCCTACCCTGCAACATTTCTGATGTWCTGAGGAAGAGTCTCCACCATAGTCCCCAAGGGCTGAGTTCTCCAGCCTGCTATCAGGGAAGGTGAGCATTGGTCCCAGGCTCTCAAAATAGTGCAGCCTCTTCTTCCCAAGCTCTGGGGTGCACCCTGTGTCCTTGGTTACCAGGAGACTAGGGTTGTGATATCTTTTCTTGTCTTGCTTTTTGATATATCAGGATTAATGTAGGAAACCAGACCTAGATTATTCAGGAGAGTAGGTATATCCCCTGTGTTTCCCAGTCTGAGTGACCAATAAAATTGTGCCTTTCTA

SEQ ID NO: 3 - protein, 4147 aa, Homo sapiens
CGCCCACTAATCCTTGATGTTCACCTTGTCCCCTGCCCCCAGAGAAGTCATCCGGACCCTCCCATCCCTGGAGTCTCTGCAGAGGTTATTTGACCAGCAGCTCTCCCCGGGCCTCCGTCCACGTCCTCAGGTTCCTGGTGAGGCCAATCCCATCAACATGGTGTCCAAGCTCAGCCAACTGACAAGCCTGTTGTCATCCATTGAAGACAAGGTCAAGGCCTTGCTGCACGAGGGTCCTGAGTCTCCGCACCGGCCCTCCCTTATCCCTCCAGTCACCTTTGAGGTGAAGGCAGAGTCTCTGGGGATTCCTCAGAAAATGCAGCTCAAAGTCGACGTTGAGTCTGGGAAACTGATCATTAAGAAGTCCAAGGATGGTTCTGAGGACAAGTTCTACAGCCACAAGAAAATCCTGCAGCTCATTAAGTCACAGAAATTTCTGAATAAGTTGGTGATCTTGGTGGAAACAGAGAAGGAGAAGATCCTGCGGAAGGAATATGTTTTTGCTGACTCCAAAAAGAGAGAAGGCTTCTGCCAGCTCCTGCAGCAGATGAAGAACAAGCACTCAGAGCAGCCGGAGCCCGACATGATCACCATCTTCATCGGCACCTGGAACATGGGTAACGCCCCCCCTCCCAAGAAGATCACGTCCTGGTTTCTCTCCAAGGGGCAGGGAAAGACGCGGGACGACTCTGCGGACTACATCCCCCATGACATTTACGTGATCGGCACCCAAGAGGACCCCCTGAGTGAGAAGGAGTGGCTGGAGATCCTCAAACACTCCCTGCAAGAAATCACCAGTGTGACTTTTAAAACAGTCGCCATCCACACGCTCTGGAACATCCGCATCGTGGTGCTGGCCAAGCCTGAGCACGAGAACCGGATCAGCCACATCTGTACTGACAACGTGAAGACAGGCATTGCAAACACACTGGGGAACAAGGGAGCCGTGGGGGTGTCGTTCATGTTCAATGGAACCTCCTTAGGGTTCGTCAACAGCCACTTGACTTCAGGAAGTGAAAAGAAACTCAGGCGAAACCAAAACTATATGAACATTCTCCGGTTCCTGGCCCTGGGCGACAAGAAGCTGAGTCCCTTTAACATCACTCACCGCTTCACGCACCTCTTCTGGTTTGGGGATCTTAACTACCGTGTGGATCTGCCTACCTGGGAGGCAGAAACCATCATCCAGAAAATCAAGCAGCAGCAGTACGCAGACCTCCTGTCCCACGACCAGCTGCTCACAGAGAGGAGGGAGCAGAAGGTCTTCCTACACTTCGAGGAGGAAGAAATCACGTTTGCCCCAACCTACCGTTTTGAGAGACTGACTCGGGACAAATACGCCTACACCAAGCAGAAAGCGACAGGGATGAAGTACAACTTGCCTTCCTGGTGTGACCGAGTCCTCTGGAAGTCTTATCCCCTGGTGCACGTGGTGTGTCAGTCTTATGGCAGTACCAGCGACATCATGACGAGTGACCACAGCCCTGTCTTTGCCACATTTGAGGCAGGAGTCACTTCCCAGTTTGTCTCCAAGAACGGTCCCGGGACTGTTGACAGCCAAGGACAGATTGAGTTTCTCAGGTGCTATGCCACATTGAAGACCAAGTCCCAGACCAAATTCTACCTGGAGTTCCACTCGAGCTGCTTGGAGAGTTTTGTCAAGAGTCAGGAAGGAGAAAATGAAGAAGGAAGTGAGGGGGAGCTGGTGGTGAAGTTTGGTGAGACTCTTCCAAAGCTGAAGCCCATTATCTCTGACCCTGAGTACCTGCTAGACCAGCACATCCTCATCAGCATCAAGTCCTCTGACAGCGACGAATCCTATGGCGAGGGCTGCATTGCCCTTCGGTTAGAGGCCACAGAAACGCAGCTGCCCATCTACACGCCTCTCACCCACCATGGGGAGTTGACAGGCCACTTCCAGGGGGAGATCAAGCTGCAGACCTCTCAGGGCAAGACGAGGGAGAAGCTCTATGACTTTGTGAAGACGGAGCGTGATGAATCCAGTGGGCCAAAGACCCTGAAGAGCCTCACCAGCCACGACCCCATGAAGCAGTGGGAAGTCACTAGCAGGGCCCCTCCGTGCAGTGGCTCCAGCATCACTGAAATCATCAACCCCAACTACATGGGAGTGGGGCCCTTTGGGCCACCAATGCCCCTGCACGTGAAGCAGACCTTGTCCCCTGACCAGCAGCCCACAGCCTGGAGCTACGACCAGCCGCCCAAGGACTCCCCGCTGGGGCCCTGCAGGGGAGAAAGTCCTCCGACACCTCCCGGCCAGCCGCCCATATCACCCAAGAAGTTTTTACCCTCAACAGCAAACCGGGGTCTCCCTCCCAGGACACAGGAGTCAAGGCCCAGTGACCTGGGGAAGAACGCAGGGGACACGCTGCCTCAGGAGGACCTGCCGCTGACGAAGCCCGAGATGTTTGAGAACCCCCTGTATGGGTCCCTGAGTTCCTTCCCTAAGCCTGCTCCCAGGAAGGACCAGGAATCCCCCAAAATGCCGCGGAAGGAACCCCCGCCCTGCCCGGAACCCGGCATCTTGTCGCCCAGCATCGTGCTCACCAAAGCCCAGGAGGCTGATCGCGGCGAGGGGCCCGGCAAGCAGGTGCCCGCGCCCCGGCTGCGCTCCTTCACGTGCTCATCCTCTGCCGAGGGCAGGGCGGCCGGCGGGGACAAGAGCCAAGGGAAGCCCAAGACCCCGGTCAGCTCCCAGGCCCCGGTGCCGGCCAAGAGGCCCATCAAGCCTTCCAGATCGGAAATCAACCAGCAGACCCCGCCCACCCCGACGCCGCGGCCGCCGCTGCCAGTCAAGAGCCCGGCGGTGCTGCACCTCCAGCACTCCAAGGGCCGCGACTACCGCGACAACACCGAGCTCCCGCATCACGGCAAGCACCGGCCGGAGGAGGGGCCACCAGGGCCTCTAGGCAGGACTGCCATGCAGTGAAGCCCTCAGTGAGCTGCCACTGAGTCGGGAGCCCAGAGGAACGGCGTGAAGCCACTGGACCCTCTCCCGGGACCTCCTGCTGGCTCCTCCTGCCCAGCTTCCTATGCAAGGCTTTGTGTTTTCAGGAAAGGGCCTAGCTTCTGTGTGGCCCACAGAGTTCACTGCCTGTGAGACTTAGCACCAAGTGCTGAGGCTGGAAGAAAAACGCACACCAGACGGGCAACAAACAGTCTGGGTCCCCAGCTCGCTCTTGGTACTTGGGACCCCAGTGCCTCGTTGAGGGCGCCATTCTGAAGAAAGGAACTGCAGCGCCGATTTGAGGGTGGAGATATAGATAATAATAATATTAATAATAATAATGGCCACATGGATCGAACACTCATGATGTGCCAAATGCTGTGCTAAGTGCTTTACGAACATTCGTCATATCAGGATGACCTCGAGAGCTGAGGCTCTAGCACCTAAAACCACGTGCCCAAACCCACCAGTTTAAAACGGTGTGTGTTCGGAGGGGTGAAAGCATTAAGAAGCCCAGTGCCCTCCTGGAGTGAGACAAGGGCTCGGCCTTAAGGAGCTGAAGAGTCTGGGTAGCTTGTTTAGGGTACAAGAAGCCTGTTCTGTCCAGCTTCAGTGACACAAGCTGCTTTAGCTAAAGTCCCGCGGGTTCCGGCATGGCTAGGCTGAGAGCAGGGATCTACCTGGCTTCTCAGTTCTTTGGTTGGAAGGAGCAGGAAATCAGCTCCTATTCTCCAGTGGAGAGATCTGGCCTCAGCTTGGGCTAGAGATGCCAAGGCCTGTGCCAGGTTCCCTGTGCCCTCCTCGAGGTGGGCAGCCATCACCAGCCACAGTTAAGCCAAGCCCCCCAACATGTATTCCATCGTGCTGGTAGAAGAGTCTTTGCTGTTGCTCCCGAAAGCCGTGCTCTCCAGCCTGGCTGCCAGGGAGGGTGGGCCTCTTGGTTCCAGGCTCTTGAAATAGTGCAGCCTTTTCTTCCTATCTCTGTGGCTTTCAGCTCTGCTTCCTTGGTTATTAGGAGAATAGATGGGTGATGTCTTTCCTTATGTTGCTTTTTCAACATAGCAGAATTAATGTAGGGAGCTAAATCCAGTGGTGTGTGTGAATGCAGAAGGGAATGCACCCCACATTCCCATGATGGAAGTCTGCGTAACCAATAAATTGTGCCTTTCTCACTCAAAACCC

SEQ ID NO: 4 - protein, 457 aa, Mus musculus
AAGCTGATGTCACTGCTCTGCAAGGAGCTCCATGGGTAACGGAGAGCCCTGAGAGAGGGGTGGGGGAGCTTCATGGGTAATGGGAGCCCCTACCCACCCAGGAGGATGGCCACAGCAAGAGAAAGTGCTCATTAGAGTGACCCTGGGTCTCCTCTCTGTCCAGATGTCTCTGCAGCACTCACAGTAATTGGCCCAGGTGGAGTCTGGAATGTTCCAGGCTTGTTGGAAGCTCTTGCTCTCATAGAATCTGAGCTCTAACTGAGCTGGGAAAGTTGATCATTTGTTTATTCCTTTTAGGGTATTGGGGGGGCACGGATGTATGCATGCTGGTATGTATGCATGCTGGGGATGTAGGGCCTCATGTGTGCTAAATACATGTGCCTATTCTGTAATGTTTTCTTGTTTGTTTTTAACTCAAATTAATTAGAGGCAGTTTCTCTGTTTACCAGCCCGGGCC

SEQ ID NO: 5 - protein, 938 aa, Mus musculus
AAATCACCACACGGCCAATACTCCCCCCCAACTCCTCCCAAATCCCCTCTACCCACTCAAATTCTTATCTTGTATTCTTTATCATTATTATACATATGTGTATATATGTGTGTGTGTATATATATATATACTATATACTGCTAATGAGTAACATTTAGTGTTATTCATTGNTGCNTGTTTNCAATGNGCTTTCCAGGNGGCTGGGGGGANGGCTCAGNGGGCAAAATTCTAGCTGCACAAGCCTAAGGACCAGGGTTCAGATCCCCAATATAAAGGCTGGCTGGACATGGGGGCTTGCCTATGATACTAGCATGCTTGCTGGAAGCAAAGACAGGGAATCCCTGGAGACTTANAATCTCANAAGTGATCTGGGCTGGACAGACTAGCTGAACTGGCCAGCTCTGGGTTCATNAANAAACCCTNCCTCCATAACATAAAGTGTGANGGANAAAGGCACCTAATGTCAACCTCAAACCCCTACCTGCATGTGCACACACATACATCCACACCACACACACACGCACACACACACACACACCACACACACACACACACAAATAAATAAGTAAATAAATAAAATATTTAGCTCTCCAGACCAAATCTTGGTGAAACCCATGCATTTGCATTTGTGTGTGTCCTACAAACACTGAAGGTTAAGAAGCATGCTCCTTAGTAATTTTATAGCAGTTTGCGTTTCCAGATTGAAAACAGATTCTATAGGCTACACAGTGCTAAATGGATTATGCTCAGATACAGATTGAAAAGGATACAGATTGAAAAGGGTCGGGGTCTGGGCCAGGATGACGGGCCAACTATCTTTGCCCGGGCTTGTCCTTCAGGGAAGGGTTACAGGATTCACCACTGGGGTGTGGCCTATCTGCTGTTAGGACCTGAATTGCCTGGAGTGTTTCTAGTTCCCACTAGTTGTTGAACTTTAC

SEQ ID NO: 6 - protein, 986 aa, Mus musculus
GCANTCTGAGGACTCTGCCATCCTGGAGTCTCTGCAGAGGTTGTTTGACCAACAGCTCTCCCCAGGCCTTCGCCCACGACCTCAGGTAAGGGGTTTGGATTTGGAAAGATGCAATTGCTATAGGAGGGACTCTGAAGGCAGACAGACGCACCGCCTCCTCACGTTGGCTAGTCTAATATAAACATCGCGGTGGATGGTGAGGATAGACTCCATGCCCTTTTGTGAAGGCATTTCCTGGCATCAGCTCCTGACTTCAGAGTTTCACCCATCAGACAAATTGCCTGGTGTTGGAGGAGGAGGTGAGCAGGGCCATTCCCATCATTTCTCCTCAGAAATGGAAAGGCAAGGAAAACATGAGGTTCTTCAGACACTTAATCCCTGGGACTGCAAAATGGTGGTGCCCCTCCTCCACAGCTGCTCACGGCGGGGCAGGAGATGAGGGCCGAATGAAGCATANATCTAGCTATTTTTTTTTTAGNGCCTTCANTAAATTTAAAATCAAATAAGGGAAGGGNCCTAAATCTTTATNTTNTGGCTTTNTAAAAGTNAAANCTTNTCCCAGGGNGCGGTGGCCCACCCCTTTNATCNACACTTGGGAGGCAAAGGCANCCGATTCTGANTTGANGCCNGCCTGGTCTNNNGNGGAGTTCCANGACANCCAGGCTTCCCANGTTTTTTTTTCACAAAACCCTTTTTTAAAACCCTNTCTAATCCNTACTCNTTNNNCCNTCANTACCATNNCANNTCCCTCTTTCNCCTNCNNTCTACTTTTNTNCCNCTNNTGNNCNATTATTTTCCTCTTCNCTTNTGTTATATNANATAATTCTCCCAACCCCTCACCCCTCTTTATTCTNANNNNNATTTTGNCATCTGCCTCCNNTTANNNTTNNTCCTCNCTTCANCTNCTTCCATTNGGTTGTNACTCNANNTTTNCTNTCTCTTANNNCTTCNCGNNNNTTCTCCANTCCNCTNNCCTNCNCCANCCC

SEQ ID NO: 7 - protein, 1001 aa, Mus musculus
GGGAGNNNNAATTAGNNAAGANANNNTTTTNTTTTNAGGACCGCNNTATGTTTATNAGAAGANNGGNGANNGANNTNTAAGGANCACCANAGNCGNCNNAAGGTTTTNAATANNGAGGNAGNCGANAAAAANANTTTNCATNTNTTTTGNCACCNNGANTTTTGAATATNANATNATGAAAANNNNTGTGTNTGNNTGTGTGTNTGTNTGTGTGNNTGTCTGTCTATNGGTGTGTGTGCNAGNGAGTGAGTAGTGAGGAGAGANAAGAAAGNATATGAGNAAAGNGCAGCTTGCNAAGAGGGATCTCTCNTACGGTGTGGGTCNCAAAGTTTGACTCCAGATTGCCATGCTTGGGGCAAATTCCTTTTNCCANAAACCATCTTGCGTGACTTNTCTANTGCTCCTGGCTTTAGGGNGGCTTTTNTGTNTGTTTTNATTTTTGTTTTCATGAGATAGGACCCCCAAATAGCCTCCTTGAGTCATACCTAAACCTTCCACCACACCCAGCTTAGCTTTTCATTTTCTAAACATGTCTGCGTAGGAATCGTCTTCCTAATTATTACCAGTCGATGTTCAATTAGCGGTTGTAACAAGGCCCCAGAGTGTGAATCTATTGCTAAGATTCTTTCCGGAAGTAGATGACACATCAGATTAATAAGTGTTGTCAAACTCATCAGTGAATGAACCATTTCCCAACAGTTCATCTGTTTCCTCCACCAAATCTGTTGGTGGAGCAGCTGGGTTACACATGGGCGAGCTGGGCTCAGCCGCGGTGCCAGCCATTTTTCCAGCATGCTATGGAATATAGGGGTGTTATCAGGCCAGGGTGGGAAAAGCCTGGGAATAGCGAGCCAGATAGCCTCACCCGGGACATTGACCATCCCAGCCATGTAGATAAATCAGAGTTGACATCACCAAAGCCAGCCACTGACTGCATGTCTTATGCTCTGAAGGTGAAGTCAGAGTCCCTGGGCATTCCTCAGAAAAGCTCCCAAAGGANG

SEQ ID NO: 8 - protein, 6498 aa, Mus musculus
GTAACGGAGAGCCCTGAGAGAGGGGTGGGGGAGCTTCATGGGTAATGGGAGCCCCTACCCACCCAGGAGGATGGCCACAGCAAGAGAAAGTGCTCATTAGAGTGACCCTGGGTCTCCTCTCTGTCCAGATGTCTCTGCAGCACTCACAGTAATTGGCCCAGGTGGAGTCTGGAATGTTCCAGGCTTGTTGGAAGCTCTTGCTCTCATAGAATCTGAGCTCTAACTGAGCTGGGAAAGTTGATCATTTGTTTATTCCTTTTAGGGTATTGGGGGGGCACGGATGTATGCATGCTGGTATGTATGCATGCTGGGGATGTAGGGCCTCATGTGTGCTAAATACATGTGCCTATTCTGTAATGTTTTCTTGTTTGTTTTTAACTCAAATTAATTAGAGGCAGTTTCTCTGTTAAAAAAAAAAAGGTGGAAGACACTGCCATCTCATTTGTGTTGGGACTTGAGATATATATATATATATATATATATATATATATATATATATAATTTATTTATTTATTTATTTGTGTATGTGTGTATAAGAAAGCGGGGGACACTCATGTGCCATGGTGTGTATGGGGACACACGTGTCCTGTGGGGGAACATTGTGTCAGGGGAACAAACATGTGCTGAGGGTGGGGGGATATATATGTGCCACTGTGGTGATTTGGTTGGGGGAGACATGTATGTGCTATGATACCTGTGTGTAGGACAGAGGACACACTCACACAGGAGCCTTCTTACCTGCTGAGCTGTCTCAAGCAGTCCAAGCCCTCCAGGCTGTAGCCACTCTTCCTCCTTGCTACAGTCCCACATCCAGCCAACACATAAAGGCTCTGGCAGGAAATAAATTAAATTTGCTTTGTGTGTGGGTGCTGACGGCTCAAGTCTTCCGGTGATGGTAATGTTTAAAGCAAGCCAACATCAGTTCTCCAGTGCCGAAGTTATTGAATGACTGACCAATGGGTAACACTTAGGATTTTTAAAAATTATTTTGATTGTACATTTTGAATTACATTAATCTTAAAATAAACTACAAACATAAACACACTGTGGTTGGAGCTGGGCATAGTGGCACATGCCTTTAATTCCAGAACTTGGGAGGCAGAGGCTGGCATATCTCTATGACTTTGAGGCCAGCCTGGTCTATATAATGAGTTCCAGGACAGAGAGATCCTGTCTCACAAACAGAGAAAACCCCATAACTATACTTTTATTGTACTGGTTGGTCTACAGTGTAAAGAATTGGCAAAGAATATGAAAGATTACACTGGGAAGAAACTGAAAGCCATCCAGAGTAATGAAGCAAACCCCTCACAAATGTGGGAAACATTTCACATGGGTGAGGGCTTGCAGCCAGCCTGTGCTAATTTATGTTTTGGTACGTGAGCACTTAGAGCAGTTTCCAGTTCTCTGGTGCTCTACCAATCTTAGCTTGGTTAATATTCAGGGATGAGTCTTCCATCACCGGTAATCTAATTTGCCATTCTATTTAAAGGCTCTTAAAGGCACAGGCAGTGCATTGGGTAAATGTGGCAAATAATTTCTTTGACAAATCGACCAATTGTCAGATTGGCCTGCTAGTCATTTGTTTCAATGAGAACTGTTTTTTCTCAAAGGATGCTCTTGTACACCGGCTAGAAGCAGGGCTGTCATTTTTATAGGTCTCTGTGGTATTTTGTTGTTGTTGTTCTGTTGTTGCAAATCATTTATCACTGAGGGAAAATACACACAAAGGGCCCTTTCTTTAAAAGTATACATGTATCATTTTGTGACAGCTCATAAGAAGCTGTTTTTTTCTGCCTGGACACAGGTCCTGACCTGTGCTGTGTCCTTGCTAAGCTTTGTCAGACCCTTCCACAGCTCCCCCCAACAACGAGTTCCCCAGTACCTGCCTCACCTCATCACTATGGTGACAGCAGCCTCTGATGCGCTGACTCTCTGGCACATTATGGCAGTGTTAAAAGCTTCCATCTCTCTCTTTGCTGAATAATGAACCTCAGGTTGTTCAAGAGACCGGAATGTTCTTCACCTGCCTGCACACATCTCTTCACTTTCTTTTATAGATCAGGTAGGGACTGGGCGTGTAGATGGAACAAACTGTTTTCCGTTCCCCAGCCATCTCTGCAGGTGCACTCCACATAAATCAAGTGTTAAAAGTGCTTTGATTAAACAGGACAGGCGCGTTCTTGAGTTCATCTGTTCACATACTGTCTGGCAAGCGCTGACTGAGGGTCTCCTCTGTACCCTGTTCTGAGAACTAACAAAAGACGAATCAACATACAGAAAACTGTTATTTAGTGACTGATTAAACTAACGAAGGCATGGGCTGGAGAAATAACTCAGCAGTTAAGAACATTTGCTGATCTTGCAGAGGACCTGGGTTGGGTTCCTAGCACACACAGGGACAGTTCCAGTCCCGGTGTGTCCTTTTCTCACTTCTGTGGACACAAGTTTTACACATAGTGCACACACATACACTCACATATATAAAACAGAACATTTAAAAGTATGTTTAAATAACGGAATCATTTATATAGGTTTTCATTTACATAGGTAAATAGGCAAAAATCTGCATTTTATTGTTTCTAAGTTTTAATTTATTTTTCTCTGTGTGTACATACGCATGCCTCCTTATCTGTATGTGCGTGCACTGTGTGCATGCATGAACCCACAGAGACCAGAAGAGTACCACAGATTCTCTGGAGCTGGAGTGATTGATAGGCTGTTGGGAGCCACTCCACATGGGGATTCAGAGTTGAACTTCGTTCTCTGCAAGAACAGCCAGCTCTTAACTGATGGCTTTTACCTCCAGCCACCTTTTCCTCATTTTTAAAATTTCCTTCCTTCCTTTTTGAGACAGGGTCTCAATACTTAGCTCATCCCAACTTGACCCCACTCTTCTCTTGCCTTAGTCACCACAATGTTTAGTTTATAAGCATGCGTCACTATGCCCGGCTTTAAATAAACTCACCCATAATCCCAGCACTGAAGTAGACAAAAGGGAGGATCGATGGGGCTGACTGGCCACAAGCCGTGCTTCAAGTTCAATGAAGACCCTGTCTCAAGGGAATAAGGCACAGAGGATAGAGCCATACGCCTGACCTCCTCCTCTGGCCTCTACCCAGGCACATGTGCATACACACACCACACACACACACACACACACACACACACACACAGAGAGAGAGAGAGAGAGAGAGAGAGAGAGAGAGAGAGAGAGAGAGAAACTTTTTCCTCTTTTTTTTTAAAAATATTATTTATTTCATGTATATGAGTACACTGTTGCTGTCTTCAGACACCCCAAAAGAAGGCATCAGATCCCATTGACAAATGGTTGTGAGCCACCATGTGGTTGCTGGGAATTGAACTCAGGACCTCTGGAAAAGCAGTCAGTGCTCTTAACCATCTCTTCAGCCCCACAAAGAAACTTTTAATGAGCAAATAATTGCTTCCAAGTAAATACTACTAATATATTTCTAACCATACTATACAAGGAATTATTAAAGAACGGATAATAGGAGAATAAAAAATTATAAGTCACTTTATAATGCTATCTAATCCATCTAGAACAAAAACACTGTAATAATGCAAAAGAGCGCAGTGCCTAGATTAAATAAATAAAATGCAGACCAATAAGTAAACTTTATAGCAGCACATGGAAATGACGAAATTCCTAACAAAAAGCTCAAGATGGGCAGTTTATTTAAAGTGAAATACAGGAGAAATAAAGCACAGAAAGATACTCAAAGGCATAGAAGTTAACATAGGGGGGCTGGCGAGATGGCTCTACAGGTAAGAGCACCCGACTGCTCTTCCAAAGGTCCTGAGTTCAAATCCCAGCAACCACATGGTGGCTCACAACCATCCGTAATGAGATTTGACTCCCTCTTCTGGTGTGTCTGAAGACAGCTACAGTGTACTTAACATATAATAAATAAATAAAACTTTAAAAAAAAAATTAAAGAAGTTAACATAGAAGCCCACTCAGGACCCCACTCAGTCCTAGAGTATGACATTATTATGGACATTAAAAAGAGAAAATTCAGCAGTAGTGTGCATGCACTGCATATATACACAAATCCTTGAGTTTCATACCAAATGCCTTTAGACCACTTGTGGCTCTGCAAACCTGTAATCCTAGCACTTGTGAAAAGGTCAGCTCAGGAACTTTGGGAAGGTCATGAAACTCTTGCCCCTCCAGAAGGGAGAGGCTAATTAACATTTCTCAGACCACAGGGCGGGAACCGACCTGCGGGTGGGGACAGACTGTTGCCCATTTCCAGACTAGGGAAGTCCTTGTCACCTCATTCCCTAAAGACCAATCAATTTAAAGGGTGCACTGTTCCGCCAATCATATTGTGCCTAGTTGCTGATGCTCTATTCTGCCCTTAGAAACCGTATAAAAACTAGCGAAGGGGTACCAGGGGTAACCCCCTCTCCTTCAGGTCTGGGACAATCCCACTACACTGGAACAATAATTTCCTCTGGCTTTTTGCATTGATCACAGCTCCACTTCGTGGTAAGTTAAGACTCCCTGGAGTCTTACATTGGCAAATGCAGGCAAAAGAATCCGAAACTCAAGGTCATCTAAAACTACATAGCAAGCATGCTGCTAGCCTGGGCTCCATGAGACCCTGGGGGAGGGGCAGAGGGAGACCGTTCAGAAGACAGTCAAGATGTTGCAGCAGCACAGGCAGCCTGGCCACCAGTGCTGTCACCAGACATGTTAATGTTGGAATAAAGCCTCAATCATGACTCTCCCAGTTTTATAATTGGAAATAAGAAAGGAAAGACTATAGGAACAACTGTGTTCAGAACACTATTTATAATAGCAAAGATCTCAGAGTAACCCAAACTTCTAGACATTGATTTGGGAAGATCTCTTGGCAGCTTATTTTGAAAACTTTACAATGTTAAATATGTAAAAACAAGGACAGTTTTGTTTTTTGTTTTGTTTTGTTTTGTTTTAGGGATATATATTCATATATGTATATGAATGAAAACCCAAACTTAAAATTCCCCACTATGCTTTAAAGGCTTTCTGACAATAACAGAAAGAGAAATAGAGAATCCATAAAAACTAGTTCTGAAACTATCAATAGGCTTGACACTCTTTAGCTGCCAGGAGAGCTGAATCTGAACACAGGGAACCCCACCCAGCACCCCAAATTTGGATTATTGTTTTATTTTATCTTTCCCCTACCCCCAAGACAGGGTTTCTCTGCGTGGTCCTGGCTGTCCTGGAACTCGGAGATCCTCTGCCTCTCTGCCTCTCTGCCTCTCTCTCTCTCTGCCTCTCTCTGCCTCTCTCTCTCTCTCTCTCTGCCTCTCTCTCTCTCTCTGCCCCTCGCTGCCTCTCTCTGCCTCTCTCTGCCCCTCTCTGCCCCTCTCTGCCCCTCTCTCTTCCCCTCTCTGCCTCTCTCTCTGCCCCTCTCTGCCTCTCTGCCTCTGCCTCCTGAGTGCTGGGATTTAAAGGCATCAGCCATCACTTCCAGCTTCCTTTATCATTTTAAAAAGAATTTCCTATGTGACTACTGTATTTAAATCACCACACGGCCAATACTCCCCCCCAACTCCTCCCAAATCCCCTCTACCCACTCAAATTCTTATCTTGTATTCTTTATCATTATTATACATATGTGTATATATGTGTGTGTGTATATATATATATACTATATACTGCTAATGAGTAACATTTAGTGTTATTCATTGTTGCATGTTTTCAATGTGCTTTCCAGGAGGCTGGGGGGATGGCTCAGTGGGCAAAATTCTAGCTGCACAAGCCTAAGGACCAGGGTTCAGATCCCCAATATAAAGGCTGGCTGGACATGGTGGCTTGCCTATGATACTAGCATGCTTGCTGGAAGCAAAGACAGGGAATCCCTGGAGACTTAGAATCTCAGAAGTGATCTGGGCTGGACAGACTAGCTGAACTGGCCAGCTCTGGGTTCATCAAGAAACCCTACCTCCATAACATAAAGTGTGATGGAGAAAGGCACCTAATGTCAACCTCAAACCCCTACCTGCATGTGCACACACATACATCCACACCACACACACACACACACACACACACACACACCACACACACACACACACAAATAAATAAGTAAATAAATAAAATATTTAGCTCTCCAGACCAAATCTTGGTGAAACCCATGCATTTGCATTTGTGTGTGTCCTACAAACACTGAAGGTTAAGAAGCATGCTCCTTAGTAATTTTATAGCAGTTTGCGTTTCCAGATTGAAAACAGATTCTATAGGCTACACAGTGCTAAATGGATTATGCTCAGATACAGATTGAAAAGGATACAGATTGAAAAGGGTCGGGGTCTGGGCCAGGATGACGGGCCAACTATCTTTGCCCGGGCTTGTCCTTCAGGGAAGGGTTACAGGATTCACCACTGGGGTGTGGCCTATCTGCTGTTAGGACCTGAATTGCCTGGAGTGTTTCTAGTTCCCACTAGTTGTTGAACTTTACCTTGAACCTCTGCTCCCAG

SEQ ID NO: 9 - protein, 361 aa, Homo sapiens
AATTTTCTACAGGCTATAGAGTGCAGTTTGGCTAAAGCAAAACCTAGGTACAGTCAGGGACTACACAATTCCAGTTCGCTGTGGGTTGGGAAGGGATGGGTGGGCCAGTGCTGGCAAGCCTTGATCTTTGCCCGGGCTTGTCCTTCTGGGGAGAATTACCTGCTTCTGCTGGACTGAGGGTGCCCTCATCTCTGGCTAGAGCCCGTGCTGCCATGGAAGACTCTTTCCGGTGCCCACTAAATCCTTGATGTTCACCTTGTCCCCTGCCCCCAGAGAAGTCATCCGGACCCTCCCATCCCTGGAGTCTCTGCAGAGGTTATTTGACCAGCAGCTCTCCCCGGGCCTCCGTCCACGTCCTCAG

SEQ ID NO: 10 - protein, 279 aa, Homo sapiens
GTAATGGCTGGCCCACGGGGGCGGGCAGGTGGGGGCGGCCACCAGGTGAGAGAAACAGCTCATGAGAGCGACCTCTGCCTCTGCCTGGCTGAACAAATGCCTCTGCCGCACTCACAATAATTGAGTGGTGAGCACAGCTTGGAAATCTCTGTGGTTGTTTGGAGGTCTTGTTATCATGGAATTTGAACAGTAGCTTGTGTGTGCTGCAGCTTTTTACAACTAAAGTTAATTGGAGGCCAGTTGCTCTGGGAAGCGGATGGGGTGGGAGGACATGGCCAG

SEQ ID NO: 11 - DNA, 784 bp, Mus musculus
  misc_feature (1)..(784): SHIP cDNA
ggcaatttctgagaggcaacaggcggcaggtctcagcctagagagggccctgaactactttgctggagtgtccgtcctgggagtggctgctgacccagtccaggagacccatgcctgccatggtccctgggtggaaccatggcaacatcacccgctccaaggcagaggagctactttccagagccggcaaggacgggagcttccttgtgcgtgccagcgagtccatcccccgggcctacgcactctgcgtgctgttccggaattgtgtttacacttacaggattctgcccaatgaggacgataaattcactgttcaggcatccgaaggtgtccccatgaggttcttcacgaagctggaccagctcatcgacttttacaagaaggaaaacatggggctggtgacccacctgcagtaccccgtgcccctggaggaggaggatgctattgatgaggctgaggaggacactgtagaaagtgtcatgtcaccacctgagctgcctcccagaaacattcctatgtctgccgggcccagcgaggccaaggaccttcctcttgcaacagagaacccccgagcccctgaggtcacccggctgagtctctccgagacactgtttcagcgtctacagagcatggataccagtgggcttcccgaggagcacctgaaagccatccaggattatctgagcactcagctcctcctggattccgactttttgaagacgggctccagcaacctccctcacctgaagaagctgatgtcactgctctgcaaggagctccatgg

SEQ ID NO: 12 - protein, 225 aa, Mus musculus
  MISC_FEATURE (1)..(225): SHIP amino acid sequence
MPAMVPGWNHGNITRSKAEELLSRAGKDGSFLVRASESIPRAYALCVLFRNCVYTYRILPNEDDKFTVQASEGVPMRFFTKLDQLIDFYKKENMGLVTHLQYPVPLEEEDAIDEAEEDTVESVMSPPELPPRNIPMSAGPSEAKDLPLATENPRAPEVTRLSLSETLFQRLQSMDTSGLPEEHLKAIQDYLSTQLLLDSDFLKTGSSNLPHLKKLMSLLCKELHG

SEQ ID NO: 13 - DNA, 2946 bp, Mus musculus
  misc_feature (1)..(2946): s-SHIP cDNA (partial)
gttcccactagttgttgaactttaccttgaacctctgctcccagggaagtcatcaggactctgccatccctggagtctctgcagaggttgtttgaccaacagctctccccaggccttcgcccacgacctcaggtgcccggagaggccagtcccatcaccatggttgccaaactcagccaattgacaagtctgctgtcttccattgaagataaggtcaagtccttgctgcacgagggctcagaatctaccaacaggcgttcccttatccctccggtcacctttgaggtgaagtcagagtccctgggcattcctcagaaaatgcatctcaaagtggacgttgagtctgggaaactgatcgttaagaagtccaaggatggttctgaggacaagttctacagccacaaaaaaatcctgcagctcattaagtcccagaagtttctaaacaagttggtgattttggtggagacggagaaggagaaaatcctgaggaaggaatatgtttttgctgactctaagaaaagagaaggcttctgtcaactcctgcagcagatgaagaacaagcattcggagcagccagagcctgacatgatcaccatcttcattggcacttggaacatgggtaatgcaccccctcccaagaagatcacgtcctggtttctctccaaggggcagggaaagacacgggacgactctgctgactacatcccccatgacatctatgtgattggcacccaggaggatccccttggagagaaggagtggctggagctactcaggcactccctgcaagaagtcaccagcatgacatttaaaacagttgccatccacaccctctggaacattcgcatagtggtgcttgccaagccagagcatgagaatcggatcagccatatctgcactgacaacgtgaagacaggcatcgccaacaccctgggaaacaagggagcagtgggagtgtccttcatgttcaatggaacctccttggggttcgtcaacagccacttgacttctggaagcgaaaaaaagctcaggagaaatcaaaactatatgaacatcctgcggttcctggccctgggagacaagaagctaagcccatttaacatcacccaccgcttcacccacctcttctggcttggggatctcaactaccgcgtggagctgcccacttgggaggcagaggccatcatccagaagatcaagcaacagcagtattcagaccttctggcccacgaccaactgctcctggagaggaaggaccagaaggtcttcctgcactttgaggaggaagagatcaccttcgcccccacctatcgatttgaaagactgacccgggacaagtatgcatacacgaagcagaaagcaacagggatgaagtacaacttgccgtcctggtgcgaccgagtcctctggaagtcttacccgctggtgcatgtggtctgtcagtcctatggcagtaccagtgacatcatgacgagtgaccacagccctgtctttgccacgtttgaagcgggagtcacatctcaattcgtctccaagaatggtcctggcactgtagatagccaagggcagatcgagtttcttgcatgctacgccacactgaagaccaagtcccagactaagttctacttggagttccactcaagctgcttagagagttttgtcaagagtcaggaaggagagaatgaagagggaagtgaaggagagctggtggtacggtttggagagactcttcccaagctaaagcccattatctctgaccccgagtacttactggaccagcatatcctgatcagcattaaatcctctgacagtgacgagtcctatggtgaaggctgcattgcccttcgcttggagaccacagaggctcagcatcctatctacacgcctctcacccaccatggggagatgactggccacttcaggggagagattaagctgcagacctcccagggcaagatgagggagaagctctatgactttgtgaagacagagcgggatgaatccagtggaatgaaatgcttgaagaacctcaccagccatgaccctatgaggcaatgggagccttctggcagggtccctgcatgtggtgtctccagcctcaatgagatgatcaatccaaactacattggtatggggccttttggacagcccctgcatgggaaatcaaccctgtccccagatcagcaactcacagcttggagttatgaccagctacccaaagactcctccctggggcctgggaggggggagggtcctccaacccctccctcccaaccacctctgtcgccaaagaagttttcatcttccacagccaaccgaggtccctgccccagggtgcaagaggcaagacctggggatctgggaaaggtggaagctctgctccaggaggacctgctgctgacgaagcccgagatgtttgagaacccactgtatggatccgtgagttccttccctaagctggtgcccaggaaagagcaggagtctcccaagatgctgcggaaggagcccccgccctgtccagacccaggaatctcatcacccagcatcgtgctccccaaagcccaagaggtggagagtgtcaaggggacaagcaaacaggcccctgtgcctgtccttggccccacaccccggatccgctcctttacctgttcttcttctgctgagggcagaatgaccagtggggacaagagccaagggaagcccaaggcctcagccagttcccaagccccagtgccagtcaagaggcctgtcaagccttccaggtcagaaatgagccagcagacaacacccatcccagctccacggccacccctgccagtcaagagtcctgctgtcctgcagctgcaacattccaaaggcagagactaccgtgacaacacagaactcccccaccatggcaagcaccgccaagaggaggggctgcttggcaggactgccatgcagtga

SEQ ID NO: 14 - protein, 928 aa, Mus musculus
  MISC_FEATURE (1)..(928): s-SHIP amino acid sequence (partial)
MVAKLSQLTSLLSSIEDKVKSLLHEGSESTNRRSLIPPVTFEVKSESLGIPQKMHLKVDVESGKLIVKKSKDGSEDKFYSHKKILQLIKSQKFLNKLVILVETEKEKILRKEYVFADSKKREGFCQLLQQMKNKHSEQPEPDMITIFIGTWNMGNAPPPKKITSWFLSKGQGKTRDDSADYIPHDIYVIGTQEDPLGEKEWLELLRHSLQEVTSMTFKTVAIHTLWNIRIVVLAKPEHENRISHICTDNVKTGIANTLGNKGAVGVSFMFNGTSLGFVNSHLTSGSEKKLRRNQNYMNILRFLALGDKKLSPFNITHRFTHLFWLGDLNYRVELPTWEAEAIIQKIKQQQYSDLLAHDQLLLERKDQKVFLHFEEEEITFAPTYRFERLTRDKYAYTKQKATGMKYNLPSWCDRVLWKSYPLVHVVCQSYGSTSDIMTSDHSPVFATFEAGVTSQFVSKNGPGTVDSQGQIEFLACYATLKTKSQTKFYLEFHSSCLESFVKSQEGENEEGSEGELVVRFGETLPKLKPIISDPEYLLDQHILISIKSSDSDESYGEGCIALRLETTEAQHPIYTPLTHHGEMTGHFRGEIKLQTSQGKMREKLYDFVKTERDESSGMKCLKNLTSHDPMRQWEPSGRVPACGVSSLNEMINPNYIGMGPFGQPLHGKSTLSPDQQLTAWSYDQLPKDSSLGPGRGEGPPTPPSQPPLSPKKFSSSTANRGPCPRVQEARPGDLGKVEALLQEDLLLTKPEMFENPLYGSVSSFPKLVPRKEQESPKMLRKEPPPCPDPGISSPSIVLPKAQEVESVKGTSKQAPVPVLGPTPRIRSFTCSSSAEGRMTSGDKSQGKPKASASSQAPVPVKRPVKPSRSEMSQQTTPIPAPRPPLPVKSPAVLQLQHSKGRDYRDNTELPHHGKHRQEEGLLGRTAMQ

SEQ ID NO: 15 - DNA, 178 bp, Mus musculus
  misc_feature (1)..(178): first exon of s-SHIP and adjacent intronic sequence
attgcctggagtgtttctagttcccactagttgttgaactttaccttgaacctctgctcccagggaagtcatcaggactctgccatccctggagtctctgcagaggttgtttgaccaacagctctccccaggccttcgcccacgacctcaggtaaggggtttggatttggaaagatgc

SEQ ID NO: 16 - DNA, 188 bp, Homo sapiens
  misc_feature (1)..(188): first exon of SIP-110 and flanking intronic sequences
catggaagactctttccggtgcccactaatccttgatgttcaccttgtcccctgcccccagagaagtcatccggaccctcccatccctggagtctctgcagaggttatttgaccagcagctctccccgggcctccgtccacgtcctcaggtaaagggtcttggggttgaaaaggtgagattaataccc

SEQ ID NO: 17 - DNA, 130 bp, Homo sapiens
  misc_feature (1)..(130): first exon of SIP-110
cgcccactaatccttgatgttcaccttgtcccctgcccccagagaagtcatccggaccctcccatccctggagtctctgcagaggttatttgaccagcagctctccccgggcctccgtccacgtcctcag

SEQ ID NO: 18 - DNA, 132 bp, Mus musculus
  misc_feature (1)..(132): first exon of s-SHIP
gttcccactagttgttgaactttaccttgaacctctgctcccagggaagtcatcaggactctgccatccctggagtctctgcagaggttgtttgaccaacagctctccccaggccttcgcccacgacctcag